SEQUENCE LISTING (27 sequences)

SEQ ID NO: 1 - RNA, 45 nt, Artificial Sequence
  3′-adapter (universal)
gaacacugcguuugcuggcuuugagaguucuacaguccgacgauc

SEQ ID NO: 2 - DNA, 21 bp, Artificial Sequence
  Reverse primer (universal)
gatcgtcggactgtagaactc

SEQ ID NO: 3 - DNA, 20 bp, Artificial Sequence
  Forward primer (for 5′-AspGUC)
gcggtcctcgttagtatagt

SEQ ID NO: 4 - DNA, 20 bp, Artificial Sequence
  Forward primer (for 5′-HisGUG)
gctcgccgtgatcgtatagt

SEQ ID NO: 5 - DNA, 15 bp, Artificial Sequence
  Forward primer (for 5′-LysCUU)
gcccggctagctcag

SEQ ID NO: 6 - DNA, 18 bp, Artificial Sequence
  Forward primer (for 5′-GluCUC)
tccctggtggtctagtgg

SEQ ID NO: 7 - DNA, 25 bp, Artificial Sequence
  TaqMan probe (for 5′-AspGUC)
tatccccgcctggaacactgcgttt

SEQ ID NO: 8 - DNA, 30 bp, Artificial Sequence
  TaqMan probe (for 5′-HisGUG)
tagtactctgcgttggaacactgcgtttgc

SEQ ID NO: 9 - DNA, 22 bp, Artificial Sequence
  TaqMan probe (for 5′-LysCUU)
agagcatgggactcgaacactg

SEQ ID NO: 10 - DNA, 19 bp, Artificial Sequence
  TaqMan probe (for 5′-GluCUC)
cgctcgaacactgcgtttg

SEQ ID NO: 11 - DNA, 19 bp, Artificial Sequence
  Forward primer (for 5′-GlyGCC)
gcattggtggttcagtggt

SEQ ID NO: 12 - DNA, 16 bp, Artificial Sequence
  Forward primer (for spike-in RNA/control)
gaggcaagcccgacgt

SEQ ID NO: 13 - DNA, 21 bp, Artificial Sequence
  Forward primer (for 5S rRNA/control)
tacggccataccaccctgaac

SEQ ID NO: 14 - DNA, 22 bp, Artificial Sequence
  TaqMan probe (for 5′-GlyGCC)
attctcgcctgcgaacactgcg

SEQ ID NO: 15 - DNA, 21 bp, Artificial Sequence
  TaqMan probe (for spike-in RNA/control)
gattgtccgcgaacactgcgt

SEQ ID NO: 16 - DNA, 28 bp, Artificial Sequence
  TaqMan probe (for 5S rRNA/control)
cgggtgctgtaggctttgaacactgcgt

SEQ ID NO: 17 - DNA, 48 bp, Artificial Sequence
  Forward Primer (5′-LysCUU)
gcttaatacgactcactatagcccggctagctcagtcggtagagcatg

SEQ ID NO: 18 - DNA, 34 bp, Artificial Sequence
  Reverse Primer (5′-LysCUU)
ccgacccgagtcccatgctctaccgactgagcta

SEQ ID NO: 19 - DNA, 49 bp, Artificial Sequence
  Forward Primer (5′-HisGUG)
gcttaatacgactcactatagccgtgatcgtatagtggttagtactctg

SEQ ID NO: 20 - DNA, 36 bp, Artificial Sequence
  Reverse Primer (5′-HisGUG)
ccgaccccaacgcagagtactaaccactatacgatc

SEQ ID NO: 21 - DNA, 49 bp, Artificial Sequence
  Forward Primer (5′-GlyCCC)
gcttaatacgactcactatagcattggtggttcagtggtagaattctcg

SEQ ID NO: 22 - DNA, 34 bp, Artificial Sequence
  Reverse Primer (5′-GlyCCC)
ccgacccgcaggcgagaattctaccactgaacca

SEQ ID NO: 23 - DNA, 30 bp, Artificial Sequence
  Forward Primer (HDV ribozyme)
taatacgactcactatagggcatctccacc

SEQ ID NO: 24 - DNA, 72 bp, Artificial Sequence
  Reverse Primer (HDV ribozyme)
gaaaagtggctctcccttagccatccgagtgctcggatgcccaggtcggaccgcgaggaggtggagatgccc

SEQ ID NO: 25 - RNA, 76 nt, Artificial Sequence
  tRNA (5′ LysCUU)
gcccggcuagcucagucgguagagcaugggacucuuaaucccagggucguggguucgagccccacguugggcgcca

SEQ ID NO: 26 - RNA, 75 nt, Artificial Sequence
  tRNA (5′ HisGUG)
gccgugaucguauagugguuaguacucugcguuguggccgcagcaaccucgguucgaauccgagucacggcacca

SEQ ID NO: 27 - RNA, 74 nt, Artificial Sequence
  tRNA (5′ GlyGCC)
gcauuggugguucagugguagaauucucgccugccacgcgggaggcccggguucgauucccggccaaugcacca

(19) United States
(12) Patent Application Publication
(10) Pub. No.: US 2021/0355548 A1
(43) Pub. Date: Nov. 18, 2021

(54) SPECIFIC EXPRESSION OF HALF-TRNA IN CANCERS

(71) Applicant: Thomas Jefferson University, Philadelphia, PA (US)

(72) Inventors: Yohei Kirino, Bryn Mawr, PA (US); Shozo Honda, Philadelphia, PA (US); Megumi Shigematsu, Philadelphia, PA (US)

(73) Assignee: Thomas Jefferson University, Philadephia, PA (US)

(21) Appl. No.: 17/313,524

(22) Filed: May 6, 2021

Related U.S. Application Data
(63) Continuation-in-part of application No. 16/802,107, filed on Feb. 26, 2020, now Pat. No. 11,001,897.
Application No. 16/802,107 is a continuation of application No. 15/116,452, filed on Aug. 3, 2016, now Pat. No. 10,662,480.
(60) Provisional application No. 61/935,795, filed on Feb. 4, 2014.

(51) Int. Cl. C12Q 1/6886; C12Q 1/6874

(52) U.S. Cl. CPC C12Q 1/6886; C12Q 1/6874; C12Q 2600/112; C12Q 2600/178; C12Q 2600/158

(57) ABSTRACT

The present invention relates to systems, devices and methods for diagnosing cancer. In various embodiments, the present invention provides a method for performing sequencing of 5′-htRNA in an RNA sample including obtaining a DNA library of 5′-htRNA and sequencing the DNA library of 5′-htRNAs in the RNA sample. The invention also teaches a method for quantifying 5′-htRNA half molecules in an RNA sample including (a) treating an RNA sample containing 5′-htRNAs half molecules having a 3′ cyclic phosphate with a T4 polynucleotide kinase to form 3′-dephosphrylated 5′-tRNA halves, (b) 3′-AD ligating the 3′-dephosphrylated 5′-tRNA halves to form ligated RNA products, and (c) quantifying the ligated RNA products by RT-qPCR using a plurality of target primers and probes configured to simultaneously quantify at least two cP-containing 5′-tRNA half species. The treating of step (a) is simultaneously performed as one step in a single tube with the 3′-AD ligating of step (b).

### CROSS-REFERENCE TO RELATED APPLICATIONS

This application is a Continuation-in-Part of U.S. patent application Ser. No. 16/802,107, filed Feb. 26, 2020 and issued as U.S. Pat. No. 11,001,897 on May 11, 2021, which is a Continuation of U.S. National Phase application Ser. No. 15/116,452, filed Aug. 3, 2016 and issued as U.S. Pat. No. 10,662,480 on May 26, 2020, which is a National Phase of International Application No. PCT/US2015/14421, filed Feb. 4, 2015, which claims priority U.S. Provisional Patent Application No. 61/935,795, filed on Feb. 4, 2014, which are incorporated herein by reference in their entirety.

### FIELD OF INVENTION

The present invention generally relates to the field of medicine and cancer. More specifically, this invention relates to systems, devices and methods for diagnosing cancer.

### BACKGROUND

All publications herein are incorporated by reference to the same extent as if each individual publication or patent application were specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

A non-coding RNA (ncRNA) is a functional RNA molecule that is not translated into a protein. Less-frequently used synonyms are non-protein-coding RNA (npcRNA), non-messenger RNA (nmRNA) and functional RNA (fRNA). The DNA sequence from which a non-coding RNA is transcribed is often called an RNA gene. Non-coding RNA genes include highly abundant and functionally important RNAs, such as transfer RNA (tRNA) and ribosomal RNA (rRNA), as well as RNAs such as snoRNAs, microRNAs, siRNAs, snRNAs, exRNAs, and piRNAs, and the long ncRNAs, such as Xist and HOTAIR. The number of ncRNAs encoded within the human genome is unknown, however recent transcriptomic and bioinformatic studies suggest the existence of thousands of ncRNAs.

During the last decade, significant attention has been directed towards the identification of novel small non-coding RNAs (sncRNAs). Recently, sncRNAs derived from tRNAs were identified as functional molecules, and not as by-products from random degradation (See Phizicky, E. M. and A. K. Hopper, tRNA biology charges to the front Genes Dev, 2010. 24(17): p. 1832-60; Sobala, A. and G. Hutvagner, Transfer RNA-derived fragments: origins, processing, and functions Wiley Interdiscip Rev RNA, 2011. 2(6): p. 853-62; Maute, R. L., et al., tRNA-derived microRNA modulates proliferation and the DNA damage response and is down-regulated in B cell lymphoma. Proc Natl Acad Sci USA, 2013. 110(4): p. 1404-9; and Lee, Y. S., et al. A novel class of small RNAs: tRNA-derived RNA fragments (tRFs). Genes Dev, 2009. 23(22): p. 2639-49, each of which is incorporated herein by reference in their entirety as though fully set forth).

There is a need in the art for diagnostic and therapeutic technologies based upon newly discovered ncRNAs and their respective functions.

### SUMMARY

The following embodiments and aspects thereof are described and illustrated in conjunction with systems, compositions and methods which are meant to be exemplary and illustrative, not limiting in scope.

Various embodiments of the present invention provide a method for quantifying a 5′-htRNA in an RNA sample. In some embodiments, the method includes (a) treating the RNA sample with a polynucleotide kinase; (b) adding a 3′-RNA adaptor to the RNA sample; (c) treating the RNA sample with an RNA ligase; (d) adding an oligonucleotide probe targeting the boundary between the 5′-htRNA and the 3′-RNA adaptor to the RNA sample; (e) performing a quantitative RT-PCR (qRT-PCR) on the RNA sample; and (f) quantifying the 5′-htRNA in the RNA sample by detecting the qRT-PCR product. The present invention also teaches a nucleic acid generated according to this method.

In certain embodiments, the present invention provides a kit. In some embodiments, the kit includes a polynucleotide kinase; a 3′-RNA adaptor; an RNA ligase; an oligonucleotide probe targeting the boundary between a 5′-htRNA and the 3′-RNA adaptor; and instructions for using the kit to quantify the 5′-htRNA in a sample.

Various embodiments of the present invention provide a method for quantifying a 3′-htRNA in an RNA sample. In some embodiments, the method includes (a) treating the RNA sample with a polynucleotide kinase; (b) adding a 5′-RNA adaptor to the RNA sample; (c) treating the RNA sample with an RNA ligase; (d) adding an oligonucleotide probe targeting the boundary between the 5′-RNA adaptor and the 3′-htRNA to the RNA sample; (e) performing a quantitative RT-PCR (qRT-PCR) on the RNA sample; and (f) quantifying the 3′-htRNA in the RNA sample by detecting the qRT-PCR product. In some embodiments, the invention provides a nucleic acid generated according to this method.

In further embodiments, the present invention provides a kit. In some embodiments, the kit includes a polynucleotide kinase; a 5′-RNA adaptor; an RNA ligase; an oligonucleotide probe targeting the boundary between the 5′-RNA adaptor and a 3′-htRNA; and instructions for using the kit to quantify the 3′-htRNA in a sample.

Various embodiments of the present invention provide a method for obtaining a DNA library of 3′-htRNAs in an RNA sample. In some embodiments, the method includes (a) treating the RNA sample with a polynucleotide kinase; (b) disrupting 3′-OH ends in the RNA sample with NaIO₄ oxidation; (c) deacylating the RNA sample with a buffer having a pH value of at least 9.0; (d) adding a 3′-RNA adaptor to the RNA sample; (e) treating the RNA sample with an RNA ligase; (f) adding a 5′-RNA adaptor to the RNA sample; (g) treating the RNA sample with an RNA ligase; and (h) performing a RT-PCR on the RNA sample, thereby obtaining the DNA library of 3′-htRNAs in the RNA sample. In some embodiments, the invention provides a DNA library of 3′-htRNAs obtained by this method.

Various embodiments of the present invention provide a method for determining the presence or absence of a cancer cell in a biological sample. In some embodiments, the method includes (a) obtaining an RNA sample from the biological sample; (b) quantifying an htRNA in the RNA sample; and (c) determining the presence of a cancer cell in the biological sample if the quantified htRNA is more than a reference value of the htRNA quantity, or determining the absence of a cancer cell in the biological sample if the quantified htRNA is not more than a reference value of the htRNA quantity.

Various embodiments of the present invention provide a method of diagnosing cancer in a subject. In some embodiments, the invention includes (a) obtaining a biological sample from the subject; (b) obtaining an RNA sample from the biological sample; (c) quantifying an htRNA in the RNA sample; and (d) diagnosing that the subject has cancer if the quantified htRNA is more than a reference value of the htRNA quantity, or diagnosing that the subject does not have cancer if the quantified htRNA is not more than a reference value of the htRNA quantity.

Various embodiments of the present invention provide a method of prognosing cancer in a subject. In some embodiments, the invention includes (a) obtaining a biological sample from the subject; (b) obtaining an RNA sample from the biological sample; (c) quantifying an htRNA in the RNA sample; and (d) prognosing that the subject is likely to develop cancer if the quantified htRNA is more than a reference value of the htRNA quantity, or prognosing that the subject is not likely to develop cancer if the quantified htRNA is not more than a reference value of the htRNA quantity.

Various embodiments of the present invention provide a method for performing sequencing of 5′-htRNA in an RNA sample. In some embodiments, the method includes obtaining a DNA library of 5′-htRNA and sequencing the DNA library of 5′-htRNAs in the RNA sample. The DNA library of 5′-htRNA may be obtained by (a) treating an RNA sample containing 5′-htRNAs having a 3′ cyclic phosphate with a phosphatase, (b) treating the phosphatase treated RNA sample with a periodate, (c) treating the periodate treated RNA sample with a polynucleotide kinase, (d) adding a 3′-RNA adaptor to the RNA sample of step c, (e) treating the RNA sample of step d with an RNA ligase, (f) adding a 5′-RNA adaptor to the RNA sample of step e, (g) treating the RNA sample of step f with an RNA ligase, and (h) performing a RT-PCR on the RNA sample of step g.

In some embodiments, the method includes enriching one or more 25-55 nt RNA fragments in the RNA sample prior to step (a). In some embodiments, the method includes gel-purifying one or more 25-55 nt RNA fragments in the RNA sample prior to step (a). In some embodiments, the method includes the 5′-htRNA is 5′-htRNA^{Asp} or 5′-htRNA^{His}. In some embodiments, the RNA sample is total RNA. In some embodiments, the RNA sample is derived from a cell, tissue, body fluid, or organ. In some embodiments, the method includes the RNA sample is approximately at least 100 pg. In some embodiments, the method includes the polynucleotide kinase is a T4 polynucleotide kinase. In some embodiments, the method includes the RNA ligase of steps (e) and (g) is a T4 RNA ligase. In some embodiments, the method includes a DNA library of 5′-htRNAs obtained by the method as described above.

Various embodiments of the present invention provide a method for quantifying 5′-htRNA half molecules in an RNA sample. In some embodiments, the method includes (a) treating an RNA sample containing 5′-htRNAs half molecules having a 3′ cyclic phosphate with a T4 polynucleotide kinase to form 3′-dephosphrylated 5′-tRNA halves, (b) 3′-AD ligating the 3′-dephosphrylated 5′-tRNA halves to form ligated RNA products, and (c) quantifying the ligated RNA products by RT-qPCR using a plurality of target primers and probes configured to simultaneously quantify at least two cP-containing 5′-tRNA half species. In some embodiments, the method includes wherein treating of step (a) is simultaneously performed as one step in a single tube with the 3′-AD ligating of step (b).

In some embodiments, the method includes that the quantifying of step (c) simultaneously quantifies 5′-tRNA half species including 5′-tRNA^{LysCUU}, 5′-tRNA^{GluCUC}, 5′-tRNA^{HisGUG}, 5′-tRNA^{GlyCCC} or combinations thereof. In some embodiments, the method includes that the quantifying of step (c) further simultaneously quantifies control RNA. In some embodiments, the control RNA includes spike-in RNA, 5S rRNA, or combinations thereof. In some embodiments, the method includes that the quantifying of step (c) simultaneously quantifies one of at least three 5′-tRNA half species and at least four 5′-tRNA half species. In some embodiments, the method includes an incubation time for the simultaneous treating and 3′-AD ligating is at most 2 h. In some embodiments, the method includes the incubation time for the simultaneous treating and 3′-AD ligating is at most 1 h at 37° C., followed by at most 1 h at 4° C. In some embodiments, the RNA sample is total RNA. In some embodiments, the RNA sample is derived from a cell, tissue, body fluid, or organ. In some embodiments, the method includes the RNA sample is approximately at least 50 ng.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 demonstrates, in accordance with an embodiment of the invention, a schematic depiction of htRNAs and uses thereof.

FIGS. 2A-2B demonstrate, in accordance with an embodiment of the invention, sequences of htRNAs derived from tRNA^{Asp} and tRNA^{His}. The arrow heads indicate the border between 5′-htRNA^{Asp} and 3′-htRNA^{Asp} and the border between 5′-htRNA^{His} and 3′-htRNA^{His}. Sequences of htRNA^{Asp} (FIG. 2A) and htRNA^{His} (FIG. 2B) were determined by RACE by using total RNA from BT474 breast cancer cells. Terminal structures of htRNAs were determined by a combination of NaIO₄ oxidation/β-elimination reaction, phosphatase and kinase treatments, and deacylation reactions as previously described in Kirino, Y. and Z. Mourelatos, Mouse Piwi-interacting RNAs are 2′-O-methylated at their 3′ termini. Nat Struct Mol Biol, 2007. 14(4): p. 347-8), which is incorporated herein by reference in its entirety as though fully set forth. It was determined that 5′-htRNAs contain a mono-phosphate (P) at their 5′-end and a cyclic-phosphate (cP) at their 3′-end, whereas 3′-htRNAs contain a hydroxyl (OH) at their 5′-end and an amino acid at their 3′-end.

FIGS. 3A-3B demonstrate, in accordance with an embodiment of the invention, an htRNA detection method. (FIG. 3A) illustrates 5′-htRNA detection: total RNA was treated with T4 PNK to remove a cyclic phosphate at the 3′-end of 5′-htRNA, and subjected to 3′-RNA adapter ligation by T4 RNA Ligase; 5′-htRNA was then detected using qRT-PCR with a Taq-Man probe targeting the boundary between the htRNA and adapter. (FIG. 3B) illustrates 3′-htRNA detection: total RNA was treated with T4 PNK to add a phosphate at the 5′-end of 3′-htRNA, and subjected to 5′-RNA adapter ligation by T4 RNA Ligase; 3′-htRNA was then detected using qRT-PCR with a Taq-Man probe targeting the boundary between the htRNA and adapter.

FIGS. 4A-4B demonstrate, in accordance with an embodiment of the invention, htRNA expression screening. (FIG. 4A) Screenings were performed for 5′-htRNA^{Asp}, 3′-htRNA^{Asp} and 5′-htRNA^{His} expressions in 96 cancer cell lines. htRNA abundance in BT20 breast cancer cells was set as 1. (FIG. 4B) The boxed portion of FIG. 4A is shown in FIG. 4B.

FIGS. 5A-5B demonstrate, in accordance with an embodiment of the invention, discovery of htRNAs in BmN4 cells. (FIG. 5A) Using Northern blots for a tRNA^{Asp}-derived piRNA, both htRNA^{Asp} and piRNA were detected. The htRNA sequence shown on the right was confirmed by RACE. The arrow heads indicate the boarders of htRNA^{Asp} and piRNA in the tRNA^{Asp}. (FIG. 5B) htRNA^{Asp} expression was reduced in thymidine-treated cells whose proliferation was arrested, suggesting a correlation between htRNA expression and cell proliferation.

FIGS. 6A-6C demonstrate, in accordance with an embodiment of the invention, htRNA expression in breast cancer. (FIG. 6A) Using Northern blots both 5′- and 3′-htRNA^{Asp} were detected in MCF7 and BT474 breast cancer cells. (FIG. 6B) htRNA expression was specifically observed in breast cancer cells. (FIG. 6C) htRNA^{Asp} and htRNA^{His} sequences were determined by RACE. 3′-htRNA^{His} was detected by Northern blot. The arrow heads indicate the border between 5′-htRNA^{Asp} and 3′-htRNA^{Asp} and the border between 5′-htRNA^{His} and 3′-htRNA^{His}.

FIGS. 7A-7B demonstrate, in accordance with an embodiment of the invention, terminal structure analyses of htRNAs. (FIG. 7A) The 5′-htRNA band detected using Northern blot was shifted up by phosphatase treatment (BAP removes P), and was even further shifted up by acid-treatment following the BAP reaction (HCl+BAP removes cyclic-P), indicating that 5′-htRNAs contain both phosphate (5′-end) and cyclic-phosphate (3′-end) at their termini. The presence of cyclic-phosphate was confirmed by the upward-shifted band by T4 Polynucleotide Kinase treatment (removes cyclic-P). miRNA-16 was used as a control. (FIG. 7B) NaIO₄ oxidation followed by β-elimination (NaIO₄, β) removed the 3′-terminal nucleotides from 3′-htRNAs only after incubation with high pH buffer (deacylation), indicating the presence of amino acids at 3′-end of 3′-htRNA. There was no change with BAP, indicating the presence of a hydroxyl terminus at the 5′-end.

FIG. 8 demonstrates, in accordance with an embodiment of the invention, selective htRNA amplification and identification. Using total RNA from BT474 breast cancer cells, 30-55 nt RNA fragments containing 5′-htRNAs with cyclic phosphate (cP), 3′-htRNAs with amino acid (AA), and other RNA species with either a phosphate (P) or a hydroxyl-terminus (OH) at their 3′-ends can be purified. To identify 5′-htRNAs, the purified RNA fraction can be deacylated and further treated with BAP, which removes AA and P, but not cP. Subsequent NaIO₄ oxidation disrupts the 3′-OH ends, and only those 5′-htRNAs with cP-blocked 3′-ends survive the treatment. These 5′-htRNAs are then treated with T4 PNK to remove cP and subsequently subjected to adapter ligations, RT-PCR and next-generation sequencing. To amplify 3′-htRNAs, the RNA fraction with T4 PNK is treated to remove P and cP, but not AA. In this case, only 3′-htRNAs, whose 3′-ends are blocked with AA, survive the subsequent NaIO₄ oxidation.

FIG. 9 demonstrates, in accordance with an embodiment of the invention, ANG mediates htRNA production. Northern blots were used to detect htRNA^{His} in BT474 cells transfected with no siRNA (Mock), a control siRNA (Control), or three different siRNAs targeting ANG (ANG 1-3). The htRNA reduction induced by ANG depletion clearly indicates the involvement of ANG in htRNA production.

FIG. 10A demonstrates In vitro production of cP-containing 5′-tRNA halves with the targeted 5′-tRNA half's regions shown in black in the cloverleaf secondary structure of respective cytoplasmic tRNAs.

FIG. 10B demonstrates In vitro synthesized HDV ribozyme and RRS-containing 5′-tRNA halves [5′-tRNA^{LysCUU} half (5′-LysCUU), 5′-tRNA^{HisGUG} half (5′-HisGUG), and 5′-tRNA^{GlyCCC} half (5′-GlyCCC)] were gel-purified and developed in denaturing PAGE.

FIG. 10C demonstrates trans-acting HDV ribozyme cleavage, the in vitro synthesized RNAs were incubated with HDV ribozyme for 10 min or 60 min. The bands of resultant cP-containing 5′-tRNA halves were observed after the cleavage reactions.

FIG. 10D demonstrates produced cP-containing 5′-tRNA halves, which were gel-purified and developed in denaturing PAGE.

FIG. 10E demonstrates 3′-terminal phosphate states of the produced 5′-tRNA halves analyzed enzymatically. The RNAs were treated with CIP or T4 PNK (NT: nontreated samples used as negative controls) and subjected to 3′-AD ligation. The ligation efficiency was estimated by quantifying the AD-ligated RNAs using TaqMan RT-qPCR. The amounts from T4 PNK-treated RNA were set as 1, and relative amounts are indicated. Averages of three technical replicates with SD values are shown.

FIG. 11A demonstrates the establishment of multiplex TaqMan RT-qPCR method for 5′-tRNA halves with a schematic representation of multiplex quantification of 5′-tRNA halves.

FIG. 11B demonstrates 10 sets of synthetic RNA mixtures to confirm the specificity of the multiplex TaqMan RT-qPCR method. The 5′-tRNA^{HisGUG} half (HEX), 5′-tRNA^{GlyCCC} half (TAMRA), and spike-in RNA (FAM) are simultaneously quantified in Group 1, and the 5′-tRNA^{LysCUU} half (HEX) and spike-in RNA (FAM) are quantified in Group 2.

FIG. 11C demonstrates an embodiment of the multiplex method being applied to the 10 sets of synthetic RNAs. The results showed specific detection of targeted RNAs by each TaqMan probe without cross-reaction. Asterisk indicates that the amplification signal was not detected.

FIG. 12A demonstrates multiplex quantification of 5′-tRNA halves using cellular total RNA with a schematic representation of multiplex quantification of 5′-tRNA halves using cellular total RNA.

FIG. 12B demonstrates total RNA from HeLa cells treated with SA or water (control: Ctrl) for 2 h were subjected to multiplex TaqMan RT-qPCR for the indicated 5′-tRNA halves. The quantified 5′-tRNA half levels were normalized to the levels of 5S rRNA. The amounts from control cells were set as 1, and relative amounts are indicated. Averages of three experiments with SD values are shown.

FIG. 13A demonstrates multiplex quantification of 5′-tRNA halves using plasma RNA with a schematic representation of the convenient version of multiplex 5′-tRNA half quantification using plasma RNA.

FIG. 13B demonstrates RNAs isolated from plasma samples of healthy individuals or Mtb-infected patients were subjected to the convenient version of multiplex TaqMan RT-qPCR for the indicated 5′-tRNA halves. The quantified 5′-tRNA half levels were normalized to the levels of spike-in RNA. The amounts from one of the healthy individuals were set as 1, and relative amounts are indicated. Averages of three experiments with SD values are shown.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul, 6(7):511-9; Queen and Selick, Humanized immunoglobulins, U.S. Pat. No. 5,585,089 (1996 Dec); and Riechmann et al., Reshaping human antibodies for therapy, Nature 1988 Mar 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For purposes of the present invention, certain terms are defined below.

“Tumor,” as used herein refers to all neoplastic cell growth and proliferation, whether malignant or benign, and all pre-cancerous and cancerous cells and tissues.

“Cancer” and “cancerous” refer to or describe the physiological condition in mammals that is typically characterized by unregulated cell growth. Examples of cancer include, but are not limited to, B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Chemotherapy resistance” as used herein refers to partial or complete resistance to chemotherapeutic drugs. For example, when a subject does not respond or only partially responds to a chemotherapeutic drug. A person of skill in the art can determine whether a subject is exhibiting resistance to chemotherapy.

“Sequence identity” is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. By way of non-limiting example, sequence identity can be calculated by software such as BLAST-P, BLAST-N, or FASTA-N, or any other appropriate software that is known in the art. The substantially identical sequences of the present invention may be at least 80%, 85%, 90%, 95%, or 100% identical to sequences described herein.

“Treated” or “treatment” as used herein in the context of an assay means applying an effective amount of a substance under conditions that allow for the action of the substance. For example, “treating a sample with ligase” means applying a sufficient amount of ligase and under the appropriate conditions (buffers, temperature, etc.) to allow for ligation, as would be recognized by one of skill in the art.

Alkaline phosphatase (ALP, ALKP) (EC 3.1.3.1) is a hydrolase enzyme responsible for removing phosphate groups from many types of molecules, including nucleotides, proteins, and alkaloids. As the name suggests, alkaline phosphatases are most effective in an alkaline environment. It is sometimes used synonymously as basic phosphatase. Examples of alkaline phosphatase include, but are not limited to, bacterial alkaline phosphatase (BAP) and calf intestinal phosphatase (CIP).

By way of background, half-tRNAs (htRNAs) were discovered to be a novel class of tRNA-derived sncRNAs expressed in breast and prostate cancers at levels significantly higher than the relatively small levels at which they may be found in certain other cancerous and noncancerous cells. To date, htRNAs have neither been described nor systematically studied in cancer or other diseases, potentially due to their 3′-end structures. Although htRNAs have a similar biogenesis mechanism as that of tRNA-derived stress-induced RNAs (tiRNAs) (See Ivanov, P., et al., Angiogenin-induced tRNA fragments inhibit translation initiation. Mol Cell, 2011. 43(4): p. 613-23; Emara, M. M., et al., Angiogenin-induced tRNA-derived stress-induced RNAs promote stress-induced stress granule assembly. J Biol Chem, 2010. 285(14): p. 10959-68; Yamasaki, S., et al., Angiogenin cleaves tRNA and promotes stress-induced translational repression. J Cell Biol, 2009. 185(1): p. 35-42; and Fu, H., et al., Stress induces tRNA cleavage by angiogenin in mammalian cells. FEBS Lett, 2009. 583(2): p. 437-42), which are produced by ANG-mediated anticodon cleavage, several characteristics (e.g., tRNA source, expression patterns of both 5′- and 3′-halves, and association not with stress but with hormone receptors) indicate htRNAs to be novel sncRNAs.

5′-htRNAs and 3′-htRNAs contain cyclic phosphates and amino acids at their 3′-ends, respectively. Such 3′-end modifications would inhibit adapter ligation, a step in normal RNA-sequencing methods; consequently, htRNAs would not be detected by traditional RNA-sequencing. As htRNAs are not abundantly detected in the publicly available sequencing datasets in breast cancer cells, the discoveries presented herein shed light on hidden layers of sncRNA biology.

As demonstrated herein in various embodiments and reported experiments, a sensitive and convenient system of detecting htRNAs from a small quantity of RNA sample was established. This detection system could be widely used for htRNA expression analyses in various samples, including patient cells, tissues, body fluids, or organs. Therefore, htRNAs can be used as biomarkers for cancer patients. As demonstrated herein, htRNA expression in cancer cells was screened, revealing that htRNAs are expressed in relatively high levels in breast and prostate cancers, but not in the other tested cancer cells. Moreover, htRNA expression in breast cancer is associated with the estrogen receptor (ER) signaling pathway, suggesting that htRNAs are key factors in cancer pathogenesis. Because htRNAs are expressed in relatively high levels in breast and prostate cancers, and their expression is correlated with hormone receptor expression, htRNAs could be used as biomarkers for diagnosis and prognosis of breast and prostate cancers. Also, htRNAs could be targets for novel therapeutic applications.

Methods for Quantifying a 5′-htRNA

In various embodiments, the present invention provides a method for quantifying a 5′-htRNA in an RNA sample. In some embodiments, the method includes (a) treating the RNA sample with a polynucleotide kinase; (b) adding a 3′-RNA adaptor to the RNA sample; (c) treating the RNA sample with an RNA ligase; (d) adding an oligonucleotide probe targeting the boundary between the 5′-htRNA and the 3′-RNA adaptor to the RNA sample; (e) performing a quantitative RT-PCR (qRT-PCR) on the RNA sample; and (f) quantifying the 5′-htRNA in the RNA sample by detecting the qRT-PCR product. In some embodiments, the invention provides a nucleic acid generated according to this method.

In further embodiments, the present invention provides a kit. In certain embodiments, the kit includes a polynucleotide kinase; a 3′-RNA adaptor; an RNA ligase; an oligonucleotide probe targeting the boundary between a 5′-htRNA and the 3′-RNA adaptor; and instructions for using the kit to quantify the 5′-htRNA in a sample.

In various embodiments described herein, the 5′-htRNA is 5′-htRNA^{Asp} or 5′-htRNA^{His}.

In various embodiments, the RNA sample is total RNA. In certain embodiments, the RNA sample is derived from a cell, tissue, body fluid, or organ. In some embodiments, the RNA sample is derived from a cancerous cell, tissue, body fluid, or organ. In certain embodiments, the RNA sample is approximately at least 1 ng. In certain embodiments, the RNA sample is approximately 1-100 or 100-1000 ng. In certain embodiments, the RNA sample is approximately at least 100 pg.

In various embodiments, the polynucleotide kinase is a T4 polynucleotide kinase. In some embodiments, the RNA ligase is a T4 RNA ligase. In various embodiments, the oligonucleotide probe is a TaqMan probe. One of skill in the art would readily appreciate that kinases, ligases and probes with similar functions as those specifically listed are contemplated within the invention.

Methods for Quantifying a 3′-htRNA

In various embodiments, the present invention provides a method for quantifying a 3′-htRNA in an RNA sample. In certain embodiments, the method includes (a) treating the RNA sample with a polynucleotide kinase; (b) adding a 5′-RNA adaptor to the RNA sample; (c) treating the RNA sample with an RNA ligase; (d) adding an oligonucleotide probe targeting the boundary between the 5′-RNA adaptor and the 3′-htRNA to the RNA sample; (e) performing a quantitative RT-PCR (qRT-PCR) on the RNA sample; and (f) quantifying the 3′htRNA in the RNA sample by detecting the qRT-PCR product. In some embodiments, the method provides a nucleic acid generated according to this method.

In certain embodiments, the present invention provides a kit. In some embodiments, the kit includes a polynucleotide kinase; a 5′-RNA adaptor; an RNA ligase; an oligonucleotide probe targeting the boundary between the 5′-RNA adaptor and a 3′-htRNA; and instructions for using the kit to quantify the 3′-htRNA in a sample.

In certain embodiments, the 3′-htRNA is 3′-htRNA^{Asp}.

In various embodiments, the RNA sample is total RNA. In some embodiments, the RNA sample is derived from a cell, tissue, body fluid, or organ. In certain embodiments, the RNA sample is derived from a cancerous cell, tissue, body fluid, or organ. In certain embodiments, the RNA sample is approximately at least 1 ng. In certain embodiments, the RNA sample is approximately 1-100 or 100-1000 ng. In certain embodiments, the RNA sample is approximately at least 100 pg.

In various embodiments, the polynucleotide kinase is a T4 polynucleotide kinase. In various embodiments, the RNA ligase is a T4 RNA ligase. In various embodiments, the oligonucleotide probe is a TaqMan probe. One of skill in the art would readily appreciate that kinases, ligases and probes with similar functions as those specifically listed are contemplated within the invention.

Methods for Obtaining a DNA Library of 5′-htRNAs

Various embodiments of the present invention provide a method for obtaining a DNA library of 5′-htRNAs in an RNA sample. In some embodiments, the method includes (a) treating an RNA sample containing 5′-htRNAs having a 3′ cyclic phosphate with a phosphatase, (b) treating the phosphatase treated RNA sample with a periodate, (c) treating the periodate treated RNA sample with a polynucleotide kinase, (d) adding a 3′-RNA adaptor to the RNA sample of step c, (e) treating the RNA sample of step d with an RNA ligase, (f) adding a 5′-RNA adaptor to the RNA sample of step e, (g) treating the RNA sample of step f with an RNA ligase, and (h) performing a RT-PCR on the RNA sample of step g. In some embodiments, the method provides a DNA library of 5′-htRNAs obtained by this method.

In various embodiments, the method further includes enriching one or more 25-55 nt RNA fragments in the RNA sample prior to step (a). In some embodiments, the method further includes gel-purifying one or more 25-55 nt RNA fragments in the RNA sample prior to step (a). In additional embodiments, the method further includes sequencing the DNA library of 5′-htRNAs in the RNA sample.

Methods for Obtaining a DNA Library of 3′-htRNAs

Various embodiments of the present invention provide a method for obtaining a DNA library of 3′-htRNAs in an RNA sample. In some embodiments, the method includes (a) treating the RNA sample with a polynucleotide kinase; (b) disrupting 3′-OH ends in the RNA sample with NaIO₄ oxidation; (c) deacylating the RNA sample with a buffer having a pH value of at least 9.0; (d) adding a 3′-RNA adaptor to the RNA sample; (e) treating the RNA sample with an RNA ligase; (f) adding a 5′-RNA adaptor to the RNA sample; (g) treating the RNA sample with an RNA ligase; and (h) performing a RT-PCR on the RNA sample, thereby obtaining the DNA library of 3′-htRNAs in the RNA sample. In some embodiments, the invention also provides a DNA library of 3′-htRNAs obtained by this method.

In some embodiments, the method further includes enriching one or more 25-55 nt RNA fragments in the RNA sample prior to step (a). In some embodiments, the method further includes gel-purifying one or more 25-55 nt RNA fragments in the RNA sample prior to step (a). In certain embodiments, the method further includes sequencing the DNA library of 3′-htRNAs in the RNA sample.

### Methods for Determining the Presence or Absence of a Cancer Cell

Various embodiments of the present invention provide a method for determining the presence or absence of a cancer cell in a biological sample. In some embodiments, the method includes (a) obtaining an RNA sample from the biological sample; (b) quantifying an htRNA in the RNA sample; and (c) determining the presence of a cancer cell in the biological sample if the quantified htRNA is more than a reference value of the htRNA quantity, or determining the absence of a cancer cell in the biological sample if the quantified htRNA is not more than a reference value of the htRNA quantity. In various embodiments, the cancer cell is a prostate cancer cell or a breast cancer cell. In various embodiments, the cancer cell is a luminal-type breast cancer cell.

In some embodiments, the biological sample is a cell, tissue, organ, blood, serum, urine, saliva, lymph, plasma, semen, or a combination thereof.

In various embodiments, the htRNA referenced in this section is 5′-htRNA or 3′-htRNA. In various embodiments, the htRNA referenced in this section is 5′-htRNA^{Asp}, 5′-htRNA^{His}, or 3′-htRNA^{Asp}.

In some embodiments, the htRNA in the RNA sample is quantified according to a method described herein for quantifying a 5′-htRNA. In other embodiments, the htRNA in the RNA sample is quantified according to a method described herein for quantifying a 3′-htRNA.

Methods of Diagnosing and/or Prognosing Cancer

Various embodiments of the present invention provide a method of diagnosing cancer in a subject. In some embodiments, the method includes (a) obtaining a biological sample from the subject; (b) obtaining an RNA sample from the biological sample; (c) quantifying an htRNA in the RNA sample; and (d) diagnosing that the subject has cancer if the quantified htRNA is more than a reference value of the htRNA quantity, or diagnosing that the subject does not have cancer if the quantified htRNA is not more than a reference value of the htRNA quantity.

Various embodiments of the present invention provide a method of prognosing cancer in a subject. In some embodiments, the method includes (a) obtaining a biological sample from the subject; (b) obtaining an RNA sample from the biological sample; (c) quantifying an htRNA in the RNA sample; and (d) prognosing that the subject is likely to develop cancer if the quantified htRNA is more than a reference value of the htRNA quantity, or prognosing that the subject is not likely to develop cancer if the quantified htRNA is not more than a reference value of the htRNA quantity.

In various embodiments, the cancer detected, diagnosed, or prognosed using the inventive methods is prostate cancer or breast cancer. In various embodiments, the cancer is luminal-type breast cancer. In some embodiments, the subject is a human. In some embodiments, the subject is a mammal. In certain embodiments, the subject is a monkey, ape, dog, cat, cow, horse, goat, pig, rabbit, mouse or rat.

In various embodiments, the biological sample is a cell, tissue, organ, blood, serum, urine, saliva, lymph, plasma, semen, or a combination thereof.

In various embodiments, the htRNA described in this section is 5′-htRNA or 3′-htRNA. In various embodiments, the htRNA is 5′-htRNA^{Asp}, 5′-htRNA^{His}, or 3′-htRNA^{Asp}.

In other embodiments, the htRNA in the RNA sample is quantified according to a method described herein for quantifying a 5′-htRNA. In some embodiments, the htRNA in the RNA sample is quantified according to a method described herein for quantifying a 3′-htRNA.

Reference Values of htRNA

In various embodiments, the reference value of an htRNA quantity is the median or mean value of the htRNA quantity in a biological sample having no cancer cell. In various embodiments, the reference value of an htRNA quantity is the median or mean value of the htRNA quantity in a biological sample having no prostate cancer cell or breast cancer cell. In various embodiments, the reference value of an htRNA quantity is the median or mean value of the htRNA quantity in a biological sample having no luminal-type breast cancer cell. In accordance with the present invention, the number of biological samples used to compute a reference value can be at least 1, 2, 5, 10, 20, 30, 40, 50, 100, or 200.

In various embodiments, the reference value of an htRNA quantity is the median or mean value of the htRNA quantity in a non-cancerous cell, tissue, body fluid, or organ. In various embodiments, the reference value of an htRNA quantity is the median or mean value of the htRNA quantity in a non-breast and non-prostate cancer cell. In accordance with the present invention, the number of cells, tissues, body fluids, or organs used to compute a reference value can be at least 1, 2, 5, 10, 20, 30, 40, 50, 100, or 200.

In various embodiments, the reference value of an htRNA quantity is the median or mean value of the htRNA quantity in biological samples from a population of subjects having no cancer. In various embodiments, the reference value of an htRNA quantity is the median or mean value of the htRNA quantity in biological samples from a population of subjects having no prostate cancer or breast cancer. In various embodiments, the reference value of an htRNA quantity is the median or mean value of the htRNA quantity in biological samples from a population of subjects having no luminal-type breast cancer. In accordance with the present invention, the number of biological samples or subjects used to compute a reference value can be at least 1, 2, 5, 10, 20, 30, 40, 50, 100, or 200.

In additional embodiments, the reference value of an htRNA quantity is the htRNA quantity in a biological sample obtained from the subject at a different (for example, an earlier or later) time point, such as during diagnosis, after diagnosis, before treatment, during treatment, after treatment, or a combination thereof.

Various statistical methods, for example, a two-tailed student t-test with unequal variation, may be used to measure the difference between an htRNA quantity in a biological sample and a reference value of an htRNA quantity. Various statistical methods, for example, a two-tailed student t-test with unequal variation, may be used to measure the differences in quantities of an htRNA between a biological sample and a control sample from a normal/healthy individual, a subject having no cancer, a subject having no prostate cancer or breast cancer, or a subject having no luminal-type breast cancer. A significant difference may be determined where the p value is equal to or less than 0.05.

In various embodiments, an htRNA is determined to be more than a reference value of an htRNA quantity by at least or about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 400, 500, 600, 700, 900, or 1000%. In various embodiments, an htRNA is quantified to be more than a reference value of the htRNA quantity by at least or about 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 4-fold, 5-fold, 6-fold, 7-fold, 8-fold, 9-fold or 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold.

The present invention may be as defined in any one of the following numbered paragraphs.

1. A method for quantifying a 5′-htRNA in an RNA sample, comprising:
(a) treating the RNA sample with a polynucleotide kinase;
(b) adding a 3′-RNA adaptor to the RNA sample;
(c) treating the RNA sample with an RNA ligase;
(d) adding an oligonucleotide probe targeting the boundary between the 5′-htRNA and the 3′-RNA adaptor to the RNA sample;
(e) performing a quantitative RT-PCR (qRT-PCR) on the RNA sample; and
(f) quantifying the 5′-htRNA in the RNA sample by detecting the qRT-PCR product.

2. The method of paragraph 1, wherein the 5′-htRNA is 5′-htRNA^{Asp} or 5′-htRNA^{His}.

3. The method of paragraph 1 or 2, wherein the RNA sample is total RNA.

4. The method of paragraph 1, 2, or 3, wherein the RNA sample is derived from a cell, tissue, or organ.

5. The method of any one of paragraphs 1-4, wherein the RNA sample is derived from a cancerous cell, tissue, or organ.

6. The method of any one of paragraphs 1-5, wherein the RNA sample is approximately at least 100 pg.

7. The method of any one of paragraphs 1-6, wherein the polynucleotide kinase is a T4 polynucleotide kinase.

8. The method of any one of paragraphs 1-7, wherein the RNA ligase is a T4 RNA ligase.

9. The method of any one of paragraphs 1-8, wherein the oligonucleotide probe is a TaqMan probe.

10. A nucleic acid generated according to the method of any one of paragraphs 1-9.

11. A kit, comprising:
(a) a polynucleotide kinase;
(b) a 3′-RNA adaptor;
(c) an RNA ligase;
(d) an oligonucleotide probe targeting the boundary between a 5′-htRNA and the 3′-RNA adaptor; and
(e) instructions for using the kit to quantify the 5′-htRNA in a sample.

12. A method for quantifying a 3′-htRNA in an RNA sample, comprising:
(a) treating the RNA sample with a polynucleotide kinase;
(b) adding a 5′-RNA adaptor to the RNA sample;
(c) treating the RNA sample with an RNA ligase;
(d) adding an oligonucleotide probe targeting the boundary between the 5′-RNA adaptor and the 3′-htRNA to the RNA sample;
(e) performing a quantitative RT-PCR (qRT-PCR) on the RNA sample; and
(f) quantifying the 3′htRNA in the RNA sample by detecting the qRT-PCR product.

13. The method of paragraph 12, wherein the 3′-htRNA is 3′-htRNA^{Asp}.

14. The method of paragraph 12 or 13, wherein the RNA sample is total RNA.

15. The method of paragraph 12, 13, or 14, wherein the RNA sample is derived from a cell, tissue, or organ.

16. The method of any one of paragraphs 12-15, wherein the RNA sample is derived from a cancerous cell, tissue, or organ.

17. The method of any one of paragraphs 12-16, wherein the RNA sample is approximately at least 100 pg.

18. The method of any one of paragraphs 12-17, wherein the polynucleotide kinase is a T4 polynucleotide kinase.

19. The method of any one of paragraphs 12-18, wherein the RNA ligase is a T4 RNA ligase.

20. The method of any one of paragraphs 12-19, wherein the oligonucleotide probe is a TaqMan probe.

21. A nucleic acid generated according to the method of any one of paragraphs 12-20.

22. A kit, comprising:
(a) a polynucleotide kinase;
(b) a 5′-RNA adaptor;
(c) an RNA ligase;
(d) an oligonucleotide probe targeting the boundary between the 5′-RNA adaptor and a 3′-htRNA; and
(e) instructions for using the kit to quantify the 3′-htRNA in a sample.

23. A method for obtaining a DNA library of 5′-htRNAs in an RNA sample, comprising:
(a) treating an RNA sample containing 5′-htRNAs having a 3′ cyclic phosphate with a phosphatase;
(b) treating the phosphatase treated RNA sample with a periodate;
(c) treating the periodate treated RNA sample with a polynucleotide kinase;
(d) adding a 3′-RNA adaptor to the RNA sample of step c;
(e) treating the RNA sample of step d with an RNA ligase;
(f) adding a 5′-RNA adaptor to the RNA sample of step e;
(g) adding a 5′-RNA adaptor to the RNA sample;
(h) treating the RNA sample of step f with an RNA ligase; and
(i) performing a RT-PCR on the RNA sample of step g, thereby obtaining the DNA library of 5′-htRNAs in the RNA sample.

24. The method of paragraph 23, further comprising enriching one or more 25-55 nt RNA fragments in the RNA sample prior to step (a).

25. The method of paragraph 23 or 24, further comprising gel-purifying one or more 25-55 nt RNA fragments in the RNA sample prior to step (a).

26. The method of paragraph 23, 24, or 25, further comprising sequencing the DNA library of 5′-htRNAs in the RNA sample.

27. A DNA library of 5′-htRNAs obtained by the method of any one of paragraphs 23-26.

28. A method for obtaining a DNA library of 3′-htRNAs in an RNA sample, comprising:
(a) treating the RNA sample with a polynucleotide kinase;
(b) disrupting 3′-OH ends in the RNA sample with NaIO₄ oxidation;
(c) deacylating the RNA sample with a buffer having a pH value of at least 9.0;
(d) adding a 3′-RNA adaptor to the RNA sample;
(e) treating the RNA sample with an RNA ligase;
(f) adding a 5′-RNA adaptor to the RNA sample;
(g) treating the RNA sample with an RNA ligase; and
(h) performing a RT-PCR on the RNA sample, thereby obtaining the DNA library of 3′-htRNAs in the RNA sample.

29. The method of paragraph 28, further comprising enriching one or more 25-55 nt RNA fragments in the RNA sample prior to step (a).

30. The method of paragraph 28 or 29, further comprising gel-purifying one or more 25-55 nt RNA fragments in the RNA sample prior to step (a).

31. The method of paragraph 28, 29, or 30, further comprising sequencing the DNA library of 3′-htRNAs in the RNA sample.

32. A DNA library of 3′-htRNAs obtained by the method of any one of paragraphs 28-31.

33. A method for determining the presence or absence of a cancer cell in a biological sample, comprising:
(a) obtaining an RNA sample from the biological sample;
(b) quantifying an htRNA in the RNA sample; and
(c) determining the presence of a cancer cell in the biological sample if the quantified htRNA is more than a reference value of the htRNA quantity, or determining the absence of a cancer cell in the biological sample if the quantified htRNA is not more than a reference value of the htRNA quantity.

34. The method of paragraph 33, wherein the cancer cell is a prostate cancer cell or a breast cancer cell.

35. The method of paragraph 33 or 34, wherein the cancer cell is a luminal-type breast cancer cell.

36. The method of paragraph 33, 34, or 35, wherein the biological sample is a cell, tissue, organ, blood, serum, urine, saliva, lymph, plasma, semen, or a combination thereof.

37. The method of any one of paragraphs 33-36, wherein the htRNA is 5′-htRNA or 3′-htRNA.

38. The method of any one of paragraphs 33-37, wherein the htRNA is 5′-htRNA^{Asp}, 5′-htRNA^{His}, or 3′-htRNA^{Asp}.

39. The method of any one of paragraphs 33-38, wherein the htRNA in the RNA sample is quantified according to the method of paragraph 1.

40. The method of any one of paragraphs 33-39, wherein the htRNA in the RNA sample is quantified according to the method of any one of paragraphs 12-15.

41. A method of diagnosing cancer in a subject, comprising:
(a) obtaining a biological sample from the subject;
(b) obtaining an RNA sample from the biological sample;
(c) quantifying an htRNA in the RNA sample; and
(d) diagnosing that the subject has cancer if the quantified htRNA is more than a reference value of the htRNA quantity, or diagnosing that the subject does not have cancer if the quantified htRNA is not more than a reference value of the htRNA quantity.

42. A method of prognosing cancer in a subject, comprising:
(a) obtaining a biological sample from the subject;
(b) obtaining an RNA sample from the biological sample;
(c) quantifying an htRNA in the RNA sample; and
(d) prognosing that the subject is likely to develop cancer if the quantified htRNA is more than a reference value of the htRNA quantity, or prognosing that the subject is not likely to develop cancer if the quantified htRNA is not more than a reference value of the htRNA quantity.

43. The method of paragraph 41 or 42, wherein the cancer is prostate cancer or breast cancer.

44. The method of paragraph 41, 42, or 43, wherein the cancer is luminal-type breast cancer.

45. The method of any one of paragraphs 41-44, wherein the subject is a human.

46. The method of any one of paragraphs 41-45, wherein the biological sample is a cell, tissue, organ, blood, serum, urine, saliva, lymph, plasma, semen, or a combination thereof.

47. The method of any one of paragraphs 41-46, wherein the htRNA is 5′-htRNA or 3′-htRNA.

48. The method of any one of paragraphs 41-47, wherein the htRNA is 5′-htRNA^{Asp}, 5′-htRNA^{His}, or 3′-htRNA^{Asp}.

49. The method of any one of paragraphs 41-48, wherein the htRNA in the RNA sample is quantified according to the method of paragraph 1.

50. The method of any one of paragraphs 41-49, wherein the htRNA in the RNA sample is quantified according to the method of paragraph 12.

51. The method of any one of paragraphs 33-50, wherein the reference value of the htRNA quantity is the median or mean value of the htRNA quantity in a biological sample having no cancer cell.

52. The method of any one of paragraphs 33-50, wherein the reference value of the htRNA quantity is the median or mean value of the htRNA quantity in a biological sample having no prostate cancer cell or breast cancer cell.

53. The method of any one of paragraphs 33-50, wherein the reference value of the htRNA quantity is the median or mean value of the htRNA quantity in a biological sample having no luminal-type breast cancer cell.

54. The method of any one of paragraphs 33-50, wherein the reference value of the htRNA quantity is the median or mean value of the htRNA quantity in a non-cancerous cell, tissue, or organ.

55. The method of any one of paragraphs 33-50, wherein the reference value of the htRNA quantity is the median or mean value of the htRNA quantity in a non-breast and non-prostate cancer cell.

56. The method of any one of paragraphs 33-50, wherein the reference value of the htRNA quantity is the median or mean value of the htRNA quantity in biological samples from a population of subjects having no cancer.

57. The method of any one of paragraphs 33-50, wherein the reference value of the htRNA quantity is the median or mean value of the htRNA quantity in biological samples from a population of subjects having no prostate cancer or breast cancer.

58. The method of any one of paragraphs 33-50, wherein the reference value of the htRNA quantity is the median or mean value of the htRNA quantity in biological samples from a population of subjects having no luminal-type breast cancer.

59. A method for quantifying 5′-htRNA half molecules in an RNA sample comprising: (a) treating an RNA sample containing 5′-htRNAs half molecules having a 3′ cyclic phosphate with a T4 polynucleotide kinase to form 3′-dephosphrylated 5′-tRNA halves; (b) 3′-AD ligating the 3′-dephosphrylated 5′-tRNA halves to form ligated RNA products; wherein treating of step (a) is simultaneously performed as one step in a single tube with the 3′-AD ligating of step (b); and (c) quantifying the ligated RNA products by RT-qPCR using a plurality of target primers and probes configured to simultaneously quantify at least two cP-containing 5′-tRNA half species.

60. The method of paragraph 59, wherein quantifying of step (c) simultaneously quantifies 5′-tRNA half species including 5′-tRNA^{LysCUU}, 5′-tRNA^{GluCUC}, 5′-tRNA^{HisGUG}, 5′-tRNA^{GlyCCC} or combinations thereof.

61. The method of paragraph 59 or 60, wherein quantifying of step (c) further simultaneously quantifies control RNA.

62. The method of any one of paragraphs 59-61, wherein the control RNA includes spike-in RNA, 5S rRNA, or combinations thereof.

63. The method of any one of paragraphs 59-62, wherein quantifying of step (c) simultaneously quantifies one of at least three 5′-tRNA half species and at least four 5′-tRNA half species.

64. The method of any one of paragraphs 59-63, wherein an incubation time for the simultaneous treating and 3′-AD ligating is at most 2 h.

65. The method of any one of paragraphs 59-64, wherein the incubation time for the simultaneous treating and 3′-AD ligating is at most 1 h at 37° C., followed by at most 1 h at 4° C.

66. The method of any one of paragraphs 59-65, wherein the RNA sample is total RNA.

67. The method of any one of paragraphs 59-66, wherein the RNA sample is derived from a cell, tissue, body fluid, or organ.

68. The method of any one of paragraphs 59-67, wherein the RNA sample is approximately at least 50 ng.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Discovery of htRNAs

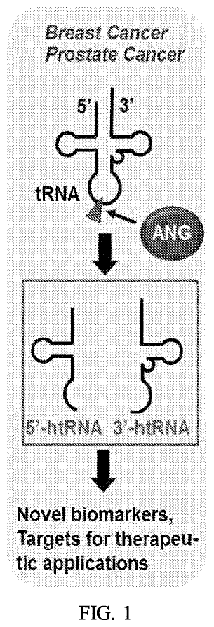

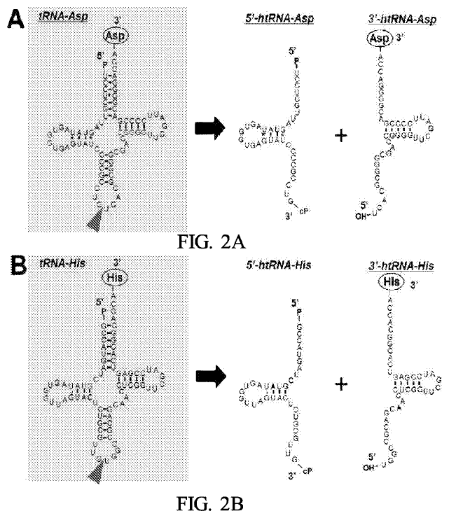

Gene expression during cancer development is controlled by a wide array of regulatory molecules, including small non-coding RNAs (sncRNAs), such as microRNAs. Certain embodiments of the present invention are based upon the discovery that htRNAs (a novel type of sncRNA derived from tRNAs) are expressed in breast and prostate cancers. htRNAs are 35-50 nucleotides (nt) long, and generated by angiogenin (ANG)-mediated cleavage at the anticodon of mature tRNAs (FIG. 1). Both 5′-(5′-htRNAs) and 3′-halves (3′-htRNAs) are derived from at least cytoplasmic tRNA^{Asp} and tRNA^{His} (FIG. 2). 5′-htRNAs contain a mono-phosphate at their 5′-end and a cyclic-phosphate at their 3′-end, whereas 3′-htRNAs contain a hydroxyl at their 5′-end and an amino acid at their 3′-end (FIG. 2).

As described herein, a sensitive system for detecting htRNA expression was established, and htRNAs were determined to be expressed in relatively high levels in breast cancer and prostate cancer cells, and not in other cancer cells and non-cancerous cells tested. Moreover, htRNA abundance was associated with cell proliferation and hormone-receptor expression.

These results implicate htRNAs as novel, important factors in breast and prostate cancer pathogenesis and suggest the use of htRNAs as novel biomarkers for the two cancers.

Establishment of htRNA Detection System

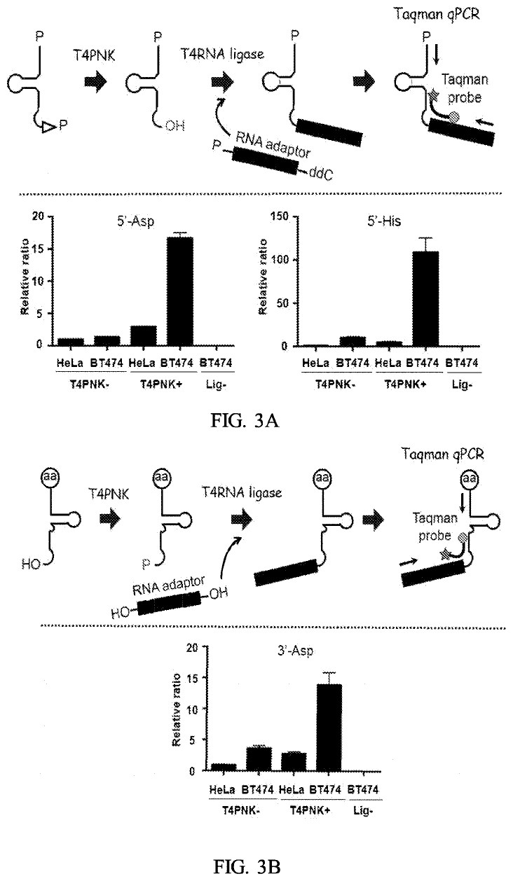

To widely screen for htRNA expression, a sensitive Taq-Man qRT-PCR-based method that detects htRNAs from about 100 pg of total RNA was established. For 5′-htRNA detection (FIG. 3A), total RNA was treated with T4 PNK to remove a cyclic phosphate at the 3′-end of 5′-htRNA, and subjected to 3′-RNA adapter ligation by T4 RNA Ligase. 5′-htRNA was then detected using qRT-PCR with a Taq-Man probe targeting the boundary between the htRNA and adapter. The inability to detect 5′-htRNA without T4 PNK or T4 RNA ligase indicated high specificity of this method. For 3′-htRNA detection (FIG. 3B), total RNA was treated with T4 PNK to add a phosphate at the 5′-end of 3′-htRNA, and subjected to 5′-RNA adapter ligation by T4 RNA Ligase. 3′-htRNA was then detected using qRT-PCR with a Taq-Man probe targeting the boundary between the htRNA and adapter. The low efficiency to detect 3′-htRNA without T4 PNK or T4 RNA ligase indicated high specificity of this method.

htRNAs are Abundantly and Specifically Expressed in Luminal-Type Breast Cancer and Prostate Cancer

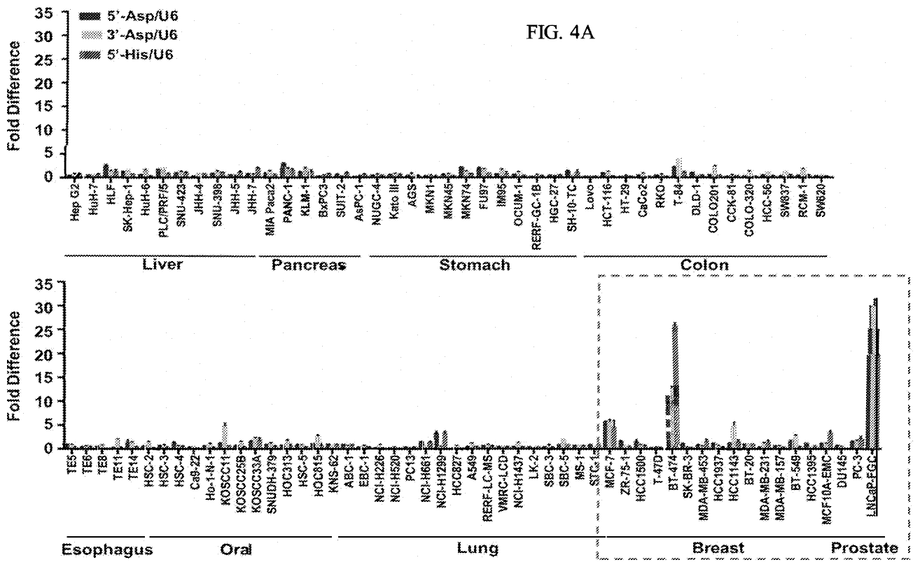

By using the detection system described herein, htRNA expression was measured in 96 cancer cell lines, revealing that htRNAs are abundantly and specifically present in luminal-type breast cancer and prostate cancer, but not in basal-like type breast cancer or other cancers (FIGS. 4A and 4B). These results suggest a relationship between hormone receptor expression and htRNA expression.

Discovery of htRNA Expression that is Associated with Cell Proliferation in BmN4 Cells

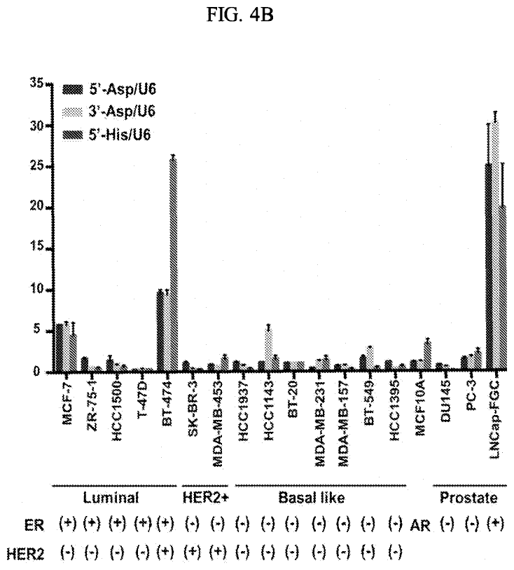

The biogenesis of piRNAs, a germline-specific class of small RNA, was investigated by taking advantage of Bombyx mori-derived BmN4 cells, as described in Honda, S., Mitochondrial protein BmPAPI modulates the length of mature piRNAs RNA, 2013. 19(10): p. 1405-18, which is incorporated herein by reference in its entirety as though fully set forth. During the analysis of a tRNA-derived piRNA, htRNAs derived from cytoplasmic tRNA^{Asp} (FIG. 5A) and tRNA^{His} (not shown) were detected. The correlation between htRNA expression and cell proliferation (FIG. 5B) suggests the expression and function of these molecules in cancer cells.

htRNA^{Asp} and htRNA^{His} are Expressed in Breast Cancer Cells

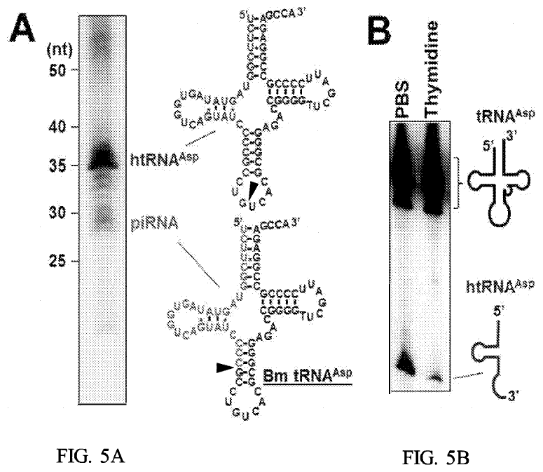

Interestingly, Northern blots revealed that both 5′- and 3′-htRNAs derived from tRNA^{Asp} and tRNA^{His} are present in MCF7 and BT474 human breast cancer cells at relatively high levels, but not in pancreas and lung cancer cells or in non-cancerous cells (FIGS. 6A and 6B). RACE showed that 5′-htRNA does not contain overlapping or intercalating sequences with 3′-htRNA (FIG. 6C), suggesting htRNA production from a single endonucleolytic cleavage. htRNAs derived from tRNA^{Ser} or tRNA^{Gly} were not detected, suggesting tRNA-specific htRNA production.

5′-htRNAs and 3′-htRNAs Contain Cyclic Phosphates and Amino Acids at their 3′-Termini, Respectively

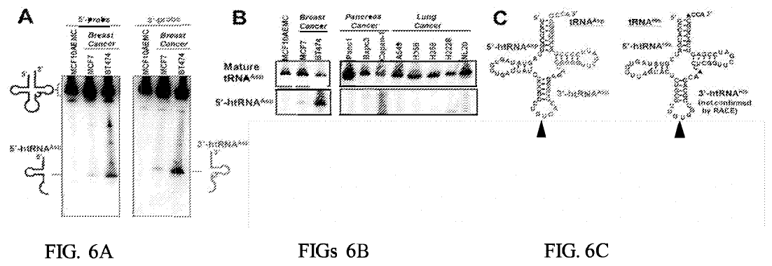

The terminal structures of htRNAs were determined using a combination of NaIO₄ oxidation/β-elimination reaction, phosphatase and kinase treatments, and deacylation reactions as previously described in Kirino, Y. and Z. Mourelatos, Mouse Piwi-interacting RNAs are 2′-O-methylated at their 3′ termini. Nat Struct Mol Biol, 2007. 14(4): p. 347-8, which is incorporated herein by reference in its entirety as though fully set forth. It was determined that 5′-htRNAs contain a mono-phosphate at their 5′-end and a cyclic-phosphate at their 3′-end, whereas 3′-htRNAs contain a hydroxyl at their 5′-end and an amino acid at their 3′-end (FIGS. 7A and 7B).

Identification of the Comprehensive htRNA Repertoire in Breast Cancer

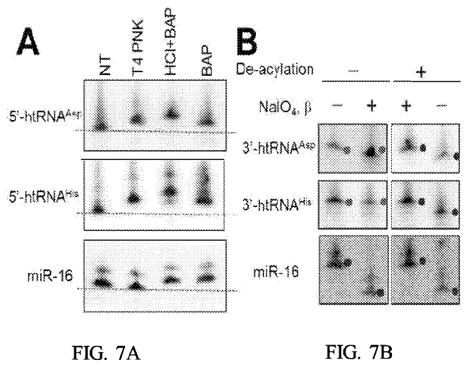

The first step towards understanding the biogenesis and precise molecular function of htRNAs in breast cancer will be to identify the complete htRNA repertoire. Utilizing the 3′-end characteristics of htRNAs, the specific species expressed in breast cancer will be selectively amplified and identified (FIG. 8). Detailed bioinformatics analyses of htRNA sequence reads will be used to confirm htRNA^{Asp} and htRNA^{His} expressions in addition to other htRNAs, and to identify the tRNA cleavage sites in htRNA biogenesis. The expression of the abundant htRNAs will be assessed in other cancer cells and patient tissues. In addition to BT474 cells, the htRNA repertoire in other htRNA-abundant breast cancer cells, such as MCF7, and in patient tissues will be further identified; the specificity, generality, and/or differences in the htRNA species and abundance will be investigated. These analyses will provide the first framework for the expression of htRNAs in cancer.

Understanding the Molecular Basis of Angiogenin-Mediated tRNA-Cleavage in htRNA Production

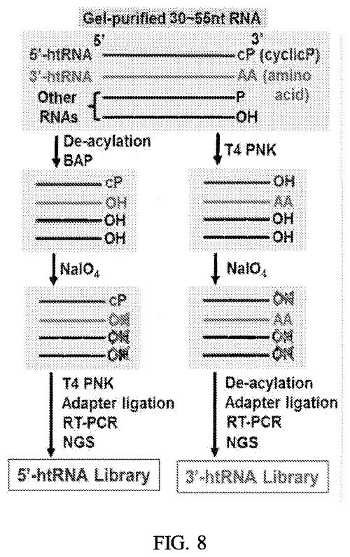

The molecular mechanisms underlying htRNA biogenesis are unknown. In mammals, tiRNAs are produced by tRNA anticodon cleavage via the ANG ribonuclease (See Ivanov, P., et al., Angiogenin-induced tRNA fragments inhibit translation initiation. Mol Cell, 2011. 43(4): p. 613-23; Emara, M. M., et al., Angiogenin-induced tRNA-derived stress-induced RNAs promote stress-induced stress granule assembly. J Biol Chem, 2010. 285(14): p. 10959-68; Yamasaki, S., et al., Angiogenin cleaves tRNA and promotes stress-induced translational repression. J Cell Biol, 2009. 185(1): p. 35-42; and Fu, H., et al., Stress induces tRNA cleavage by angiogenin in mammalian cells. FEBS Lett, 2009. 583(2): p. 437-42). The research presented in the present application demonstrates that ANG is also responsible for htRNA production in breast cancer (FIG. 9). Recombinant human ANG protein could be produced and purified. In vitro reactions will be designed, in which the ANG protein is incubated with 32P-labeled in vitro-transcribed tRNA for anticodon cleavage. Using kinetic analyses of the various tRNA species and their mutants, the positive and negative determinants of tRNA sequences necessary for htRNA production will be determined to understand the molecular basis of ANG-mediated tRNA cleavage in breast cancer.

Understanding the Mechanisms Underlying the Specific Expression of the htRNAs in Breast Cancer

htRNA expression is highly specific to breast cancer cells (FIG. 4A, FIG. 6B), and the precise molecular mechanisms behind this specificity will be investigated. While not wishing to be bound by a particular theory, some possible reasons that htRNAs are abundant in breast cancer could be (i) ANG is activated (Yamasaki, S., et al., Angiogenin cleaves tRNA and promotes stress-induced translational repression. J Cell Biol, 2009. 185(1): p. 35-42), or RNH1, an inhibitor of ANG, could be deactivated, and/or (ii) m5C methylation at position 38 of tRNA^{Asp}, which is mediated by DNMT2 and protects the tRNA from ANG-mediated anticodon cleavage, is deficient (See Goll, M. G., et al., Methylation of tRNAAsp by the DNA methyltransferase homolog Dnmt2. Science, 2006. 311(5759): p. 395-8; and Schaefer, M., et al., RNA methylation by Dnmt2 protects transfer RNAs against stress-induced cleavage. Genes Dev, 2010. 24(15): p. 1590-5). To address these hypotheses, the expression and localization of ANG, RNH1 and TRDMT1 in breast cancer cells and in other types of cancer cells will be analyzed. Furthermore, the rate of tRNAAsp-m5C38 modification in breast cancer cells will be investigated using biochemical analyses, including thin layer chromatography and the Donis-Keller method (See Kirino, Y. and Z. Mourelatos, Mouse Piwi-interacting RNAs are 2′-O-methylated at their 3′ termini. Nat Struct Mol Biol, 2007. 14(4): p. 347-8, and Kirino, Y., et al., Codon-specific translational defect caused by a wobble modification deficiency in mutant tRNA from a human mitochondrial disease. Proc Natl Acad Sci USA, 2004. 101(42): p. 15070-5, which are each incorporated herein by reference in their entirety as though full set forth), and mass-spectrometry (See Kirino, Y., et al., Acquisition of the wobble modification in mitochondrial tRNALeu (CUN) bearing the G12300A mutation suppresses the MELAS molecular defect. Hum Mol Genet, 2006. 15(6): p. 897-904), which is incorporated herein by reference in its entirety as though full set forth).

htRNAs are Abundantly and Specifically Expressed in Luminal-Type Breast Cancer and Prostate Cancer

To widely screen for htRNA expression, a sensitive Taq-Man qRT-PCR-based method that detects 5′-htRNAs from 100 pg of total RNA was established (FIG. 3A). htRNA expression was measured in 96 cancer cell lines, revealing that htRNAs are abundantly present in luminal-type breast cancer and prostate cancer (See comprehensive molecular portraits of human breast tumors. Nature, 2012. 490(7418): p. 61-70), but not in basal-like type breast cancer or other cancers.

Unraveling the Molecular Function of htRNAs in Luminal-Type Breast Cancer

The research described in this application strongly suggests an association between htRNA expression and the ER signaling pathways. The direct link will be explored by analyzing htRNA, ANG, RNH1 and DNMT2 expressions in BT474 cells with overexpressed or repressed ER and HER2. Without wishing to be bound by a particular theory, it is hypothesized that htRNAs are involved in gene expression regulation, as suggested by previous studies that described the roles for other tRNA-derived RNAs on the inhibitions of mRNA expression and translation (See Maute, R. L., et al., tRNA-derived microRNA modulates proliferation and the DNA damage response and is down-regulated in B cell lymphoma. Proc Natl Acad Sci USA, 2013. 110(4): p. 1404-9; Lee, Y.S., et al., A novel class of small RNAs: tRNA-derived RNA fragments (tRFs). Genes Dev, 2009. 23(22): p. 2639-49; and Ivanov, P., et al., Angiogenin-induced tRNA fragments inhibit translation initiation. Mol Cell, 2011. 43(4): p. 613-23, each of which is incorporated herein by reference as though fully set forth). htRNAs in BT474 cells could be silenced using 2′-O-methylated anti-sense oligonucleotides, and (1) global translation by pulse-labeling, (2) RNA expression by RNA-sequencing, and (3) the cell proliferation, colony formation, and migration rates will be investigated. These studies will help elucidate the precise biological function of htRNAs in breast and prostate cancers.

Multiplex TaqMan RT-qPCR Method to Simultaneously Quantify Multiple cP-Containing 5′-tRNA Half Species

RNA cleavages by many ribonucleases generate RNA molecules that contain a 2′,3′-cyclic phosphate (cP) at their 3′-termini, and many cP-containing RNAs (cP-RNAs) are expressed as functional molecules in cells and tissues. 5′-tRNA half molecules are representative examples of functional cP-RNAs, playing important roles in various biological processes. Various embodiments herein use in vitro production of cP-containing 5′-tRNA half molecules that prepare abundant synthetic cP-RNAs enough for functional analyses. Various embodiments include further a multiplex TaqMan RT-qPCR method that simultaneously quantifies multiple cP-containing 5′-tRNA half species. This multiplex method provides efficient quantification of 5′-tRNA halves using samples with limited amounts, such as human body fluid samples, e.g., human plasma samples, revealing drastic enhancement of 5′-tRNA half levels at approximately 1000-fold in patients infected with Mycobacterium tuberculosis. These in vitro production and multiplex quantification methods can be applied to any cP-RNAs, while providing cost-effective, in-house techniques to accelerate expressional and functional characterizations of 5′-tRNA halves and other cP-RNAs.

In biogenesis of non-coding RNA (ncRNA) molecules, newly transcribed RNAs usually undergo multiple maturation steps in which enzymatic cleavages of the RNAs play crucial roles. RNA cleavages by many ribonucleases generate RNA molecules that contain a 2′,3′-cyclic phosphate (cP) at their 3′-termini [1]. The 3′-terminal cP end is also formed by ribozyme cleavages or is generated de novo by the enzymes, such as RtcA and MthRnl, that convert a 3′-terminal phosphate (P) to a cP [1]. Many cP-containing RNAs (cP-RNAs) have been demonstrated to be expressed as functional molecules. In some embodiments, examples of functional cP-RNAs include the 5′-tRNA half molecules produced from tRNA anticodon cleavage by angiogenin (ANG), a member of the RNase A super family [1]. In mammalian cells, ANG cleavage of tRNAs is induced by various biological phenomenon, such as stress stimuli, the sex hormone signaling pathway, and mycobacterial infection [2-6]. The resultant cP-containing 5′-tRNA half molecules play important roles in a variety of biological processes. Stress-induced 5′-tRNA halves promote stress granule formation, regulate translation, and trigger cellular stress responses and apoptosis in neurodevelopmental disorders [2, 7-10]. Sex hormone-dependent 5′-tRNA halves promote cell proliferation in hormone-dependent breast and prostate cancers [4, 11]. Infection-induced 5′-tRNA halves promote immune response by activating Toll-like receptor 7 (TLR7) [5]. 5′-tRNA halves further serve as direct precursors of Piwi-interacting RNAs (piRNAs) in germ cells [12]. Because cP-RNAs cannot be ligated to a 3′-adapter (AD) in standard RNA-seq procedure, cP-RNAs are not captured by standard RNA-seq, forming a hidden component in the transcriptome [1]. A cP-RNA-seq was developed that is able to specifically sequence cP-RNAs [4, 13] and recently performed genome-wide identification of short cP-RNA transcriptome, revealing abundant expression of numerous cP-RNAs derived not only from tRNAs but also from rRNAs and mRNAs [6, 14].

To further expand cP-RNA research, it is imperative to continuously unravel expressional regulations and biological roles of cP-RNAs. Previously, functional characterizations of 5′-tRNA halves relied on the transfection of cP-lacking synthetic RNAs into cultured cells. However, cP formation is not just the consequence of specific ribonuclease digestions—cP formation itself could have a functional significance: cP formation regulates RNA-protein interaction and RNA stability [15-19], and it is required for specific ligation reaction [20, 21]. Recent discovery of cP-specific phosphatase further suggests the importance of cP-formation [22]. Thus, although cP-RNA syntheses are relatively costly and not widely offered, it is preferable to utilize synthetic cP-RNAs, instead of cP-lacking RNAs, for functional analyses of cP-RNAs. Utilization of synthetic cP-RNAs also contributes to unraveling fundamental roles of a cP. Disclosed herein is in vitro production of cP-containing 5′-tRNA half molecules that is able to prepare abundant synthetic cP-RNAs, which are sufficient for performing functional analyses. Furthermore, given that the expression levels of 5′-tRNA half molecules are drastically changed in various biological processes and diseases [2, 4-6, 23], a specific cP-RNA quantification method fulfills a long-felt need in cP-RNA research.

Various embodiments herein disclose a multiplex TaqMan RT-qPCR method that efficiently, specifically, and simultaneously quantifies multiple cP-containing 5′-tRNA half species. These methods provide cost-effective, in-house techniques to accelerate characterizations of 5′-tRNA halves and other cP-RNAs.

In Vitro Production of cP-Containing 5′-tRNA Halves

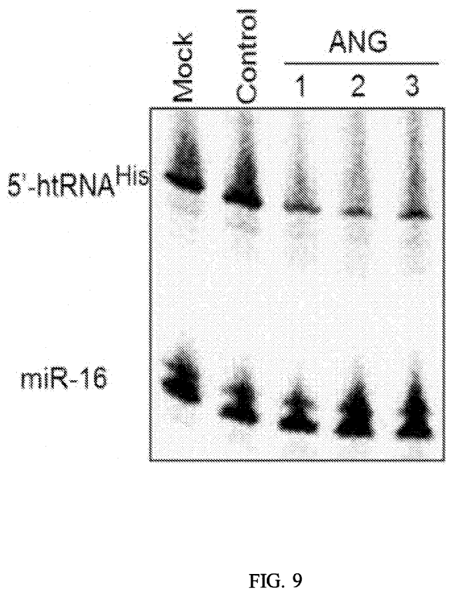

To illustrate the multiplex method, the sequences of three cP-containing 5′-tRNA halves half) synthesized (5′-tRNA^{LysCUU} half, 5′-tRNA^{HisGUG} half, and 5′-tRNA^{GlyCCC} half) synthesized are shown in FIG. 10A. Three cP-RNAs were produced according to the Ciesiolka's method [24] with modifications. The method consists of the two steps: 1) in vitro synthesis of RNAs containing a 3′-terminal hepatitis delta virus (HDV) ribozyme recognition site (RRS: 5′-GGGUCGG-3′); and 2) 3′-end cleavage of the synthesized RNAs by trans-acting HDV ribozyme to form a cP end. In vitro RNA synthesis was performed as described previously [5, 25]. dsDNA templates were synthesized using the primers shown in Table S1. The templates were then subjected to an in vitro transcription reaction with T7 RNA polymerase (New England Biolabs) at 37° C. for 4 h. The synthesized RNAs were then gel-purified using denaturing PAGE with single-nucleotide resolution. The in vitro synthesized RRS-containing 5′-tRNA half (3 μM) and HDV ribozyme (6 μM) were subjected to annealing procedure by incubating in 18 μl mixture containing 2 μl of 10× reaction buffer [500 mM Tris-HCl (pH 7.5) and 1 mM EDTA] at 90° C. for 2 min, followed by incubation at 0° C. for 10 min and then at 37° C. for 10 min. Subsequently, RNA cleavage by HDV ribozyme was started by adding 2 μl of 100 mM MgCl₂ solution and incubating at 37° C. for 60 min. The cleaved RNAs were then gel-purified using denaturing PAGE.

The sequences as shown in FIG. 10A include: (i) for 5′-tRNA^{LysCUU} GCCCGGCUAGCUCAGUCGGUAGAGCAUGGGACUCUUAAUCCCAGGGUCGUGGG UUCGAGCCCCACGUUGGGCGCCA (SEQ ID NO: 25); (ii) 5′-tRNA^{HisGUG} GCCGUGAUCGUAUAGUGGUUAGUACUCUGCGUUGUGGCCGCAGCAACCUCGGU UCGAAUCCGAGUCACGGCACCA (SEQ ID NO: 26); and (iii) for 5′-tRNA^{GlyCCC} GCAUUGGUGGUUCAGUGGUAGAAUUCUCGCCUGCCACGCGGGAGGCCCGGGUU CGAUUCCCGGCCAAUGCACCA (SEQ ID NO: 27).

Confirmation of a cP End in the Produced 5′-tRNA Halves

The 3′-terminal phosphate states of the produced 5′-tRNA halves were analyzed as described previously [11, 13, 14]. The RNAs were treated with calf intestinal phosphatase (CIP; New England Biolabs) or T4 polynucleotide kinase (T4 PNK; New England Biolabs). The RNAs were then subjected to specific quantification of 5′-tRNA halves using 3′-AD ligation and TaqMan RT-qPCR as described previously [5, 7, 11, 13, 14].

Multiplex TaqMan RT-qPCR for Quantification of Synthetic 5′-tRNA Halves

Various embodiments of the multiplex method provide for quantifying 5′-tRNA halves in an RNA sample. In various embodiments, the biological sample is a cell, tissue, body fluid, or organ, or a combination thereof. Body fluid may include blood, serum, urine, saliva, lymph, plasma, semen, or a combination thereof.

The produced cP-containing 5′-tRNA halves (100 fmol each), mixed with 500 ng of E. coli total RNA, were treated with 10 units of T4 PNK in 10 μl reaction mixture at 37° C. for 1 h. One μl of the mixture was then added to a 3′-AD ligation mixture (total 10 μl) containing 0.1 mg/ml BSA, 1 mM ATP, 5% PEG8000, 20 pmol of 3′-AD [4], 20 units of RNase inhibitor (Promega), 10 units of T4 RNA ligase (T4 Rnl: Thermo Scientific), and 1×T4 Rnl reaction buffer. For 3′-AD ligation, the mixture was incubated at 37° C. for 1 h, followed by overnight incubation at 4° C. The ligated RNAs were diluted 20-fold, and 2 μl of the diluted mixture was subjected to TaqMan RT-qPCR (reaction mixture volume 10 μl) using One Step PrimeScript RT-PCR Kit (Takara) and StepOne Plus Real-time PCR machine (Applied Biosystems). Two groups of multiplex quantification were established: Group 1 for 5′-tRNA^{HisGUG} half (hexachlorofluorescein, HEX), 5′-tRNA^{GlyCCC} half (carboxytetramethylrhodamine, TAMRA), and spike-in control RNA (6-carboxyfluorescein, FAM; 5′-GGGAGGCAAGCCCGACGUCGUCCAGAUUGUCCGC-3′), and Group 2 for 5′-tRNA^{LysCUU} half (HEX) and spike-in control RNA (FAM), respectively. The sequences and concentrations of primers and TaqMan probes are shown in Table S2. The cycle condition of PCR was: 42° C. for 5 min and 95° C. for 10 s, followed by up to 40 cycles of 95° C. for 10 s and 63° C. for 34 s. While total RNA is used in this example, other RNA samples are contemplated including but not limited to fractionated RNAs and isolated RNAs and the like.

### Cell Culture, Induction of Oxidative Stress, and RNA Isolation

HeLa cells were cultured in DMEM (Life Technologies) containing 10% FBS. The cells were treated with 500 μM of sodium arsenite (SA; Sigma) to induce oxidative stress and expression of stress-induced tRNA halves as described previously [13]. Total RNA from the cells was isolated using TRIsure (Bioline).

Multiplex Quantification of 5′-tRNA Halves for Cellular RNAs

To remove a cP from cP-RNAs, total RNA was first treated with T4 PNK as described previously [4]. Then, 50 ng of the treated RNAs were subjected to 3′-AD ligation reaction, followed by multiplex TaqMan RT-qPCR as described above.

### Ethical Approval, Human Plasma Samples, and RNA Isolation

Human plasma samples from patients infected with Mycobacterium tuberculosis (Mtb) was obtained without private information and in accordance with all federal, institutional, and ethical guidelines. Plasma samples were obtained from a biological specimen company, BioIVT, without receiving patients' information. Human plasma samples were derived from healthy or Mtb-infected males aged 30-35 years. RNA isolation from the plasma samples was performed as described previously [11]. First, 500 μl of plasma was centrifuged at 16,060 g for 5 min, and then 400 μl of supernatant was mixed with 1 fmol of the spike-in control RNA and subjected to RNA extraction using TRIzol LS (Thermo Fisher Scientific). The extracted RNAs were further subjected to purification using the miRNeasy Mini Kit (Qiagen).

Multiplex Quantification of 5′-tRNA Halves for Plasma RNAs

The multiplex 5′-tRNA half quantification method as described herein was adapted to plasma RNAs by combining T4 PNK treatment and 3′-AD ligation into a one-step reaction. Plasma RNA sample (2 μl) was added to the reaction mixture (total volume 20 μl) containing 0.1 mg/ml BSA, 2 mM ATP, 5% PEG8000, 1 μM 3′-AD, 40 units of RNasin® Ribonuclease Inhibitor (Promega), 10 units of T4 Rnl, 10 units of T4 PNK, 1 mM DTT, and 1×T4 RNA ligase reaction buffer. The reaction mixture was first incubated at 37° C. for 1 h, followed by incubation at 4° C. for 1 h. The reacted mixture was diluted 3-fold, and 2 μl of the diluted mixture was subjected to the multiplex TaqMan RT-qPCR (reaction mixture volume 10 μl) as described above.

Selection of 5′-tRNA Half Species for Production And Quantification

The three 5′-tRNA halves (5′-tRNA^{LysCUU} half, 5′-tRNA^{HisGUG} half, 5′-tRNA^{GlyCCC} half, FIG. 10A) were selected because they are abundantly and differentially expressed in various cells and tissues. 5′-tRNA^{LysCUU} half and 5′-tRNA^{GlyCCC} half were the two most abundant tRNA-derived cP-RNAs in oxidative stress-induced HeLa and U2OS cells [6]. Together, the 5′-tRNA^{LysCUU} half and 5′-tRNA^{HisGUG} half occupied >87% of sex hormone-dependent tRNA-derived cP-RNAs in BT-474 breast cancer cells [4]. 5′-tRNA^{HisGUG} half and 5′-tRNA^{GlyCCC} half were among the top 4 abundant 5′-tRNA halves identified in extracellular vehicles (EVs) secreted from human monocyte-derived macrophages [5]. The EV-5′-tRNA^{HisGUG} half, which plays an important role in promoting the immune response by activating TLR7, were greatly upregulated in plasma samples from Mtb-infected patients [5]. Regarding the 5′-tRNA^{LysCUU} half and 5′-tRNA^{HisGUG} half, the molecules ranging from the 5′-end to the anticodon 1^{st} nucleotide [nucleotide position (np) 1-34 according to the nucleotide numbering system of tRNAs [26]] (FIG. 10A) were commonly the most abundant molecules in the above sequencing studies, so these molecules were targeted in this disclosure. Regarding the 5′-tRNA^{GlyCCC} half its 3′-end position showed variations in cP-RNA-seq data from various cells and tissues. In this disclosure, the 5′-tRNA^{GlyCCC} half of np 1-35 (FIG. 10A) were targeted, which accumulated the most prominently as stress-induced molecules [6] and which was the most abundant in mouse tissues [20].

In Vitro Production of cP-Containing 5′-tRNA Halves

In vitro transcription by bacteriophage T7 RNA polymerase has been a standard, widely used method to synthesize RNAs of desired sequences [27], but the synthesized RNAs possess a hydroxyl group (OH) at their 3′-end. To enable rapid, abundant, and cost-effective synthesis of cP-containing 5′-tRNA halves, the in vitro RNA synthesis was employed, followed by 3′-terminal cleavage of the RNAs using a trans-acting HDV ribozyme based on the Ciesiolka's method [24, 28]. As in the RNA cleavages catalyzed by all other self-cleaving ribozymes, HDV ribozyme cleavage forms a cP at the 3′-end of 5′-cleavage products [29]. To be cleaved by the HDV ribozyme, RNAs need to contain 7-nt RRS sequences; these sequences are recognized and hybridized by the ribozyme [24, 28]. In vitro transcription by T7 RNA polymerase successfully synthesized the three 5′-tRNA halves containing 3′-terminal RRS sequences, as well as the HDV ribozyme (FIG. 10B). After gel-purification, the RRS-containing 5′-tRNA halves were subjected to HDV ribozyme-catalyzed cleavage. Incubation of the RNAs with the HDV ribozyme produced RRS-lacking 5′-tRNA halves (FIG. 10C), which were expected to contain a cP. Compared to a 10-min incubation, an incubation of 60 min provided better yields of the cleavage products (FIG. 10C). In some embodiments, 60 min is used for incubation time for 5′-tRNA half synthesis. After gel-purification, the quality of the produced cP-containing 5′-tRNA halves as a single band in denaturing PAGE (FIG. 10D) was confirmed. To confirm the presence of a cP in the synthesized 5′-tRNA halves, the RNAs were treated with CIP (removes 3′-P) or T4 PNK (removes 3′-P/cP). The efficiency of the ligations between each RNA and a 3′-AD by TaqMan RT-qPCR was subsequently examined, as described in previous studies [4-6, 12-14], the produced 5′-tRNA halves exhibited the highest amplification signals upon T4 PNK treatment, while non-treated RNAs and CIP-treated RNAs did not yield significant amplification signals (FIG. 10E). These results indicate that the majority of the produced 5′-tRNA halves contain a cP at their 3′-ends and provide evidence of the successful production of cP-containing 5′-tRNA halves. Approximately 6.7-8.7 μg (˜600-800 pmol) of cP-containing 5′-tRNA halves were produced from 1 ml of T7 RNA polymerase reaction. This method is easy to scaled-up and is applicable to abundantly produce any short cP-RNAs.

Multiplex TaqMan RT-qPCR for Quantification of Cellular 5′-tRNA Halves

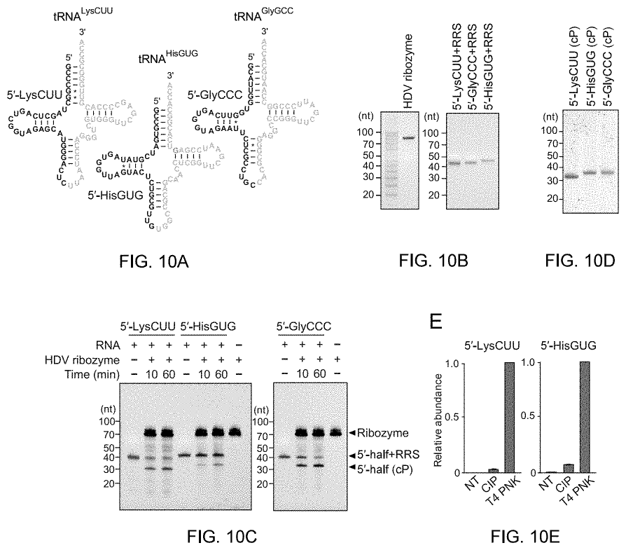

The majority of cP-RNAs are produced from abundant substrate RNAs [14]. As a result, cP-RNAs and their substrate RNAs usually coexist in the cells and in RNA. In some embodiments, the RNA is total RNA. For example, 5′-tRNA halves coexist with corresponding mature tRNAs and their precursors, which are usually much more abundant than 5′-tRNA halves. Standard RT-qPCR amplification of the interior sequences of 5′-tRNA halves cannot distinguish signals of 5′-tRNA halves and their substrate RNAs. Instead, a specific TaqMan RT-qPCR method that is able to exclusively quantify 5′-tRNA halves was developed [4] and this method was utilized for quantification of 5′-tRNA halves in human cultured cells, Bombyx cells, and mouse tissues [5, 6, 12, 14]. While the previous method was singleplex and targeted only one 5′-tRNA half per reaction, herein is disclosed an upgraded version of TaqMan RT-qPCR by employing a multiplex approach that involves three steps (FIG. 11A). First, total RNA extracted from cells or tissues with T4 PNK was treated to remove a cP from 5′-tRNA halves. Then 3′-AD were ligated to the 3′-dephosphrylated 5′-tRNA halves, followed by quantification of the ligation products by TaqMan RT-qPCR. Because TaqMan probe is designed to target the boundary of the targeted 5′-tRNA halves and 3′-AD, this method quantified only the 3′-AD-ligated 5′-tRNA half but not the corresponding mature tRNA and its precursor. To perform the TaqMan RT-qPCR, a one-step reaction of reverse transcription and qPCR quantification was conducted in the presence of multiple target primers and TaqMan probes (FIG. 11A). Two target 5′-tRNA halves were simultaneously quantified by TaqMan probes either with HEX or TAMRA, and control spike-in RNA was also detected by FAM-containing probe at the same time. This multiplex method can have two advantages over the singleplex method disclosed also herein. First, more than one target 5′-tRNA half can be simultaneously quantified, which saves effort, cost, and RNA sample for 5′-tRNA half quantification. Saving the amounts of starting RNA sample is especially important when using precious and limited samples such as those from disease patients. Second, this method allows the user to quantify 5′-tRNA halves and control RNA at the same time. Because the amounts of control RNA are used for normalization, the simultaneous quantification would enhance accuracy of the 5′-tRNA half quantification. In various embodiments, the biological sample is a cell, tissue, body fluid, or organ, or a combination thereof. Body fluid may include blood, serum, urine, saliva, lymph, plasma, semen, or a combination thereof.

To establish the multiplex method and to confirm its specificity, first used were synthetic cP-containing 5′-tRNA halves produced by the in vitro method (FIG. 10D), as well as synthetic control spike-in RNA. Not all multiplex combinations of targeted RNAs resulted in specific quantification of each target RNA without cross-reaction. Two groups of multiplex quantifications were established: Group 1 for the quantification of the 5′-tRNA^{HisGUG} half (HEX) 5′-tRNA^{GlyCCC} half (TAMRA), and spike-in RNA (FAM); and Group 2 for the quantification of the 5′-tRNA^{LysCUU} half (HEX) and spike-in RNA (FAM) (FIG. 11B). To confirm their specificities, ten sets of synthetic RNA mixtures including various combinations of the three 5′-tRNA halves and spike-in RNA (FIG. 11B) were subjected to the multiplex TaqMan RT-qPCR for Groups 1 and 2. As shown in FIG. 11C, in both Groups 1 and 2, each targeted RNA was specifically detected without being influenced by the presence of other RNAs in the mixture. The mixtures lacking the targeted RNAs did not yield amplification signals at all. These results confirmed that the multiplex method as disclosed herein is able to specifically quantify the targeted 5′-tRNA halves.

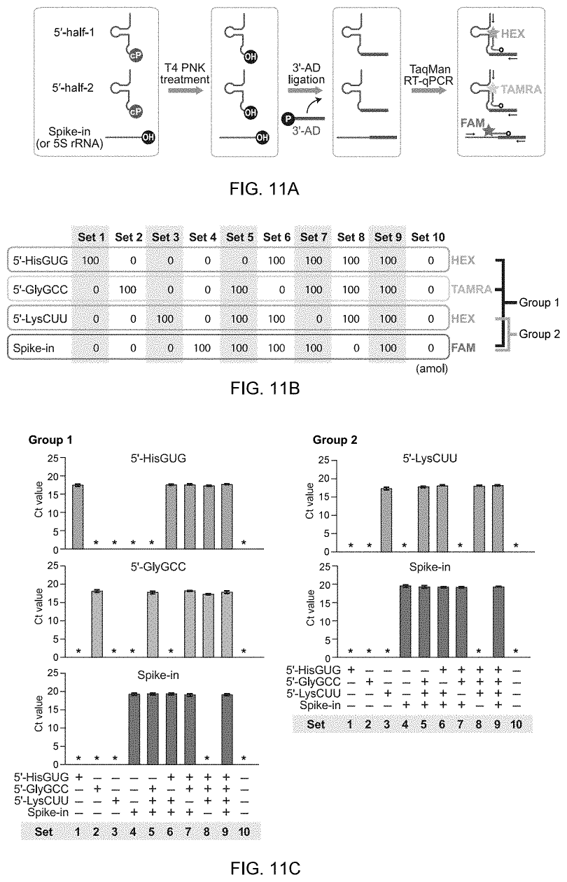

Next the applicability of the multiplex TaqMan RT-qPCR method to cellular total RNA was examined. When cellular RNAs are used as materials, instead of control spike-in RNA, the 5S rRNA is detected as a control for normalization. To illustrate cellular samples that show differential expression of 5′-tRNA halves, total RNA from SA-treated HeLa cells were utilized where the induction of oxidative stress accumulated stress-induced, cP-containing 5′-tRNA halves [6]. Total RNAs from non-treated (control) or SA-treated HeLa cells were subjected to T4 PNK treatment, followed by 3′-AD ligation and multiplex TaqMan RT-qPCR for Groups 1 and 2 (FIG. 12A). Relative abundances of the 5′-tRNA halves between non-treated and SA-treated samples were normalized by the abundance of simultaneously quantified 5S rRNA. Upregulation of all of the quantified 5′-tRNA halves (FIG. 12B) was observed, which is consistent with the previous study [6]. Upon SA treatment, the expression of the 5′-tRNA^{HisGUG} half 5′-tRNA^{GlyCCC} half and 5′-tRNA^{LysCUU} half increased 22.6-, 25.3-, and 65.2-fold (average of two biological replicates), respectively.

Multiplex TaqMan RT-qPCR for Quantification of Plasma 5′-tRNA Halves

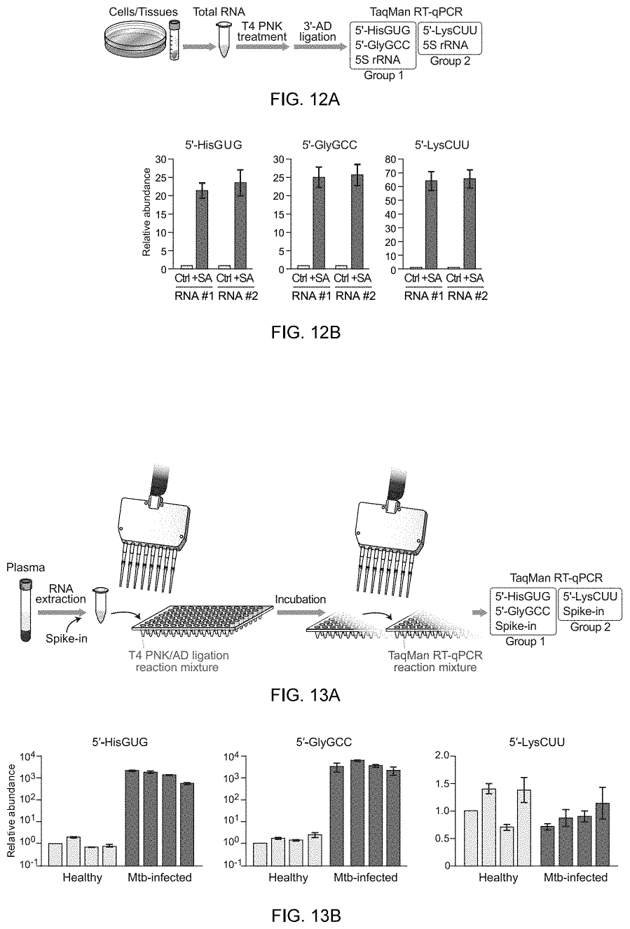

Dynamic secretion of RNA molecules and transportation of the secreted extracellular RNAs (exRNAs) into recipient cells can modulate their phenotypes, showing that exRNAs act as key transducers of intercellular communications [30-33]. These exRNAs circulate in biofluids or body fluids, and given their accessibility, abundance, and stability, coupled with their cellular roles, they are regarded as a powerful source of biomarkers for diseases [34]. This study revealed upregulation of extracellular 5′-tRNA halves (ex-5′-tRNA halves) in plasma samples from Mtb-infected patients [5], which have been utilized herein to establish a multiplex quantification method of ex-5′-tRNA halves for plasma samples. Because human tissue samples are precious and generally limited in their quantity, a more efficient and convenient version of the method for multiplex 5′-tRNA half quantification (FIG. 13A) was established by making the following modifications. First, the T4 PNK treatment and 3′-AD ligation were simultaneously performed as one step in one tube, which can reduce starting amounts of RNA samples. Second, the incubation time for the T4 PNK treatment/3′-AD ligation was shortened to 2 h (1 h at 37° C., followed by 1 h at 4° C.), which increases detection efficiency of ex-5′-tRNA halves due to less degradation during the reactions. Both modifications are important, especially when starting RNA amounts are limited. At the same time, this method requires only two sample transfers between tubes or plates, enabling the user to conveniently quantify ex-5′-tRNA halves using large numbers of samples (FIG. 13A).

To test the applicability of the method, quantified were the three ex- 5′-tRNA halves in plasma samples from healthy individuals or Mtb-infected patients. Because the expression of tRNA halves can be affected by sex hormones [4] and aging [14], the examined individuals were limited to males aged 30 to 35 years as in a previous study [5]. A synthetic spike-in RNA was added during plasma RNA extraction, and its abundance was later used for normalization. The relative abundance of the three examined ex- 5′-tRNA halves were successfully quantified by using the convenient version of multiplex TaqMan RT-qPCR (FIG. 13B). The expression levels of the ex-5′-tRNA^{HisGUG} half and ex-5′-tRNA^{GlyCCC} half were drastically enhanced approximately 1000-fold in Mtb-infected patients compared to healthy individuals, while the levels of the ex-5′-tRNA^{LysCUU} half showed similar abundance between the healthy individuals and the patients. The method disclosed herein is advantageously applicable to the samples with limited RNA quantity, such as plasma samples.

The multiplex TaqMan RT-qPCR method for 5′-tRNA half quantification as disclosed herein provides efficient and convenient quantification. Molecular function of 5′-tRNA halves/cP-RNAs can vary depending on their sequences and species, such that the 5′-tRNA^{HisGUG} half activates TLR7 whereas the 5′-tRNA^{LysCUU} half does not [5]. In various embodiments, the capture of individual cP-RNA species leads to expressional and functional characterizations for each cP-RNA. The methods of cP-RNA production and quantification described herein are widely applicable to any cP-RNAs, including recently-identified mRNA- and rRNA-derived cP-RNAs [6, 14]. Further, the multiplex method as disclosed herein can be useful to screen various biological materials and disease samples to characterize differential cP-RNA expressions. The methods disclosed herein provide much-needed techniques to produce and quantify the 5′-tRNA halves and other cP-RNAs.

[1] M. Shigematsu, T. Kawamura, Y. Kirino, Generation of 2′,3′-Cyclic Phosphate-Containing RNAs as a Hidden Layer of the Transcriptome, Front Genet 9 (2018) 562.

[2] S. Yamasaki, P. Ivanov, G. F. Hu, P. Anderson, Angiogenin cleaves tRNA and promotes stress-induced translational repression, J Cell Biol 185(1) (2009) 35-42.

[3] H. Fu, J. Feng, Q. Liu, F. Sun, Y. Tie, J. Zhu, R. Xing, Z. Sun, X. Zheng, Stress induces tRNA cleavage by angiogenin in mammalian cells, FEBS Lett 583(2) (2009) 437-42.

[4] S. Honda, P. Loher, M. Shigematsu, J. P. Palazzo, R. Suzuki, I. Imoto, I. Rigoutsos, Y. Kirino, Sex hormone-dependent tRNA halves enhance cell proliferation in breast and prostate cancers, Proc Natl Acad Sci USA 112(29) (2015) E3816-25.

[5] K. Pawar, M. Shigematsu, S. Sharbati, Y. Kirino, Infection-induced 5′-half molecules of tRNAHisGUG activate Toll-like receptor 7, PLoS Biol 18(12) (2020) e3000982.

[6] M. Shigematsu, Y. Kirino, Oxidative stress enhances the expression of 2′,3′-cyclic phosphate-containing RNAs, RNA Biol 17(8) (2020) 1060-1069.

[7] P. Ivanov, M. M. Emara, J. Villen, S. P. Gygi, P. Anderson, Angiogenin-induced tRNA fragments inhibit translation initiation, Molecular cell 43(4) (2011) 613-23.

[8] S. Blanco, S. Dietmann, J. V. Flores, S. Hussain, C. Kutter, P. Humphreys, M. Lukk, P. Lombard, L. Treps, M. Popis, S. Kellner, S. M. Holter, L. Garrett, W. Wurst, L. Becker, T. Klopstock, H. Fuchs, V. Gailus-Durner, M. Hrabe de Angelis, R. T. Karadottir, M. Helm, J. Ule, J. G. Gleeson, D. T. Odom, M. Frye, Aberrant methylation of tRNAs links cellular stress to neuro-developmental disorders, The EMBO journal 33(18) (2014) 2020-39.

[9] P. Ivanov, E. O'Day, M. M. Emara, G. Wagner, J. Lieberman, P. Anderson, G-quadruplex structures contribute to the neuroprotective effects of angiogenin-induced tRNA fragments, Proceedings of the National Academy of Sciences of the United States of America 111(51) (2014) 18201-6.

[10] M. M. Emara, P. Ivanov, T. Hickman, N. Dawra, S. Tisdale, N. Kedersha, G.F. Hu, P. Anderson, Angiogenin-induced tRNA-derived stress-induced RNAs promote stress-induced stress granule assembly, J Biol Chem 285(14) (2010) 10959-68.

[11] S. Honda, Y. Kirino, SHOT-RNAs: A novel class of tRNA-derived functional RNAs expressed in hormone-dependent cancers, Mol Cell Oncol 3(2) (2016) e1079672.

[12] S. Honda, T. Kawamura, P. Loher, K. Morichika, I. Rigoutsos, Y. Kirino, The biogenesis pathway of tRNA-derived piRNAs in Bombyx germ cells, Nucleic Acids Res 45(15) (2017) 9108-9120.

[13] S. Honda, K. Morichika, Y. Kirino, Selective amplification and sequencing of cyclic phosphate-containing RNAs by the cP-RNA-seq method, Nat Protoc 11(3) (2016) 476-89.

[14] M. Shigematsu, K. Morichika, T. Kawamura, S. Honda, Y. Kirino, Genome-wide identification of short 2′,3′-cyclic phosphate-containing RNAs and their regulation in aging, PLoS Genet 15(11) (2019) e1008469.

[15] Y. Nikolaev, C. Deillon, S. R. Hoffmann, L. Bigler, S. Friess, R. Zenobi, K. Pervushin, P. Hunziker, B. Gutte, The leucine zipper domains of the transcription factors GCN4 and c-Jun have ribonuclease activity, PLoS One 5(5) (2010) e10765.

[16] K. Licht, J. Medenbach, R. Luhrmann, C. Kambach, A. Bindereif, 3′-cyclic phosphorylation of U6 snRNA leads to recruitment of recycling factor p110 through LSm proteins, RNA 14(8) (2008) 1532-8.

[17] K. P. Lee, M. Dey, D. Neculai, C. Cao, T. E. Dever, F. Sicheri, Structure of the dual enzyme Irel reveals the basis for catalysis and regulation in nonconventional RNA splicing, Cell 132(1) (2008) 89-100.

[18] C. Zhang, C. Yin, L. Wang, S. Zhang, Y. Qian, J. Ma, Z. Zhang, Y. Xu, S. Liu, HSPC111 governs breast cancer growth by regulating ribosomal biogenesis, Mol Cancer Res 12(4) (2014) 583-94.

[19] M. B. Sporn, H. M. Lazarus, J. M. Smith, W. R. Henderson, Studies on nuclear exoribonucleases. 3. Isolation and properties of the enzyme from normal and malignant tissues of the mouse, Biochemistry 8(4) (1969) 1698-706.

[20] J. Popow, A. Schleiffer, J. Martinez, Diversity and roles of (t)RNA ligases, Cell Mol Life Sci 69(16) (2012) 2657-70.

[21] T. Yoshihisa, Handling tRNA introns, archaeal way and eukaryotic way, Front Genet 5 (2014) 213.

[22] P. H. Pinto, A. Kroupova, A. Schleiffer, K. Mechtler, M. Jinek, S. Weitzer, J. Martinez, ANGEL2 is a member of the CCR4 family of deadenylases with 2′,3′-cyclic phosphatase activity, Science 369(6503) (2020) 524-530.

[23] P. Anderson, P. Ivanov, tRNA fragments in human health and disease, FEBS Lett 588(23) (2014) 4297-304.

[24] A. Wichlacz, M. Legiewicz, J. Ciesiolka, Generating in vitro transcripts with homogenous 3′ ends using trans-acting antigenomic delta ribozyme, Nucleic acids research 32(3) (2004) e39.

[25] M. Shigematsu, Y. Kirino, 5′-Terminal nucleotide variations in human cytoplasmic tRNAHisGUG and its 5′-halves, RNA 23(2) (2017) 161-168.

[26] M. Sprinzl, C. Horn, M. Brown, A. loudovitch, S. Steinberg, Compilation of tRNA sequences and sequences of tRNA genes, Nucleic Acids Res 26(1) (1998) 148-53.

[27] J. F. Milligan, D. R. Groebe, G. W. Witherell, O. C. Uhlenbeck, Oligoribonucleotide synthesis using T7 RNA polymerase and synthetic DNA templates, Nucleic acids research 15(21) (1987) 8783-98.

[28] J. Wrzesinski, M. Legiewicz, B. Smolska, J. Ciesiolka, Catalytic cleavage of cis- and trans-acting antigenomic delta ribozymes in the presence of various divalent metal ions, Nucleic acids research 29(21) (2001) 4482-92.

[29] H. Schurer, K. Lang, J. Schuster, M. Morl, A universal method to produce in vitro transcripts with homogeneous 3′ ends, Nucleic acids research 30(12) (2002) e56.

[30] M. Baj-Krzyworzeka, R. Szatanek, K. Weglarczyk, J. Baran, B. Urbanowicz, P. Branski, M. Z. Ratajczak, M. Zembala, Tumour-derived microvesicles carry several surface determinants and mRNA of tumour cells and transfer some of these determinants to monocytes, Cancer Immunol Immunother 55(7) (2006) 808-18.

[31] H. Valadi, K. Ekstrom, A. Bossios, M. Sjostrand, J. J. Lee, J. O. Lotvall, Exosome-mediated transfer of mRNAs and microRNAs is a novel mechanism of genetic exchange between cells, Nat Cell Biol 9(6) (2007) 654-9.

[32] K. Li, R. S. Rodosthenous, F. Kashanchi, T. Gingeras, S. J. Gould, L. S. Kuo, P. Kurre, H. Lee, J. N. Leonard, H. Liu, T. B. Lombo, S. Momma, J. P. Nolan, M. J. Ochocinska, D. M. Pegtel, Y. Sadovsky, F. Sanchez-Madrid, K. M. Valdes, K. C. Vickers, A. M. Weaver, K. W. Witwer, Y. Zeng, S. Das, R. L. Raffai, T. K. Howcroft, Advances, challenges, and opportunities in extracellular RNA biology: insights from the NIH exRNA Strategic Workshop, JCI Insight 3(7) (2018).

[33] N. Sadik, L. Cruz, A. Gurtner, R. S. Rodosthenous, S. A. Dusoswa, O. Ziegler, T. S. Van Solinge, Z. Wei, A. M. Salvador-Garicano, B. Gyorgy, M. Broekman, L. Balaj, Extracellular RNAs: A New Awareness of Old Perspectives, Methods Mol Biol 1740 (2018) 1-15.

[34] K. E. A. Max, K. Bertram, K. M. Akat, K. A. Bogardus, J. Li, P. Morozov, I. Z. Ben-Dov, X. Li, Z. R. Weiss, A. Azizian, A. Sopeyin, T. G. Diacovo, C. Adamidi, Z. Williams, T. Tuschl, Human plasma and serum extracellular small RNA reference profiles and their clinical utility, Proceedings of the National Academy of Sciences of the United States of America 115(23) (2018) E5334-E5343.

Supplementary Information Table S1

TABLE S1
Sequences of DNA primer for the synthesis of dsDNA templates
for in vitro RNA synthesis

| Target | Primer Sequence (5′ to 3′) | |
|---|---|---|
| 5′-LysCUU | Forward | GCTTAATACGACTCACTATAGCCCGGCTAGCTCAGTCGGTAGAGCATG |
| | | (SEQ ID NO: 17) |
| | Reverse | CCGACCCGAGTCCCATGCTCTACCGACTGAGCTA (SEQ ID NO: 18) |
| 5′-HisGUG | Forward | GCTTAATACGACTCACTATAGCCGTGATCGTATAGTGGTTAGTACTCTG |
| | | (SEQ ID NO: 19) |
| | Reverse | CCGACCCCAACGCAGAGTACTAACCACTATACGATC (SEQ ID NO: 20) |
| 5′-GlyCCC | Forward | GCTTAATACGACTCACTATAGCATTGGTGGTTCAGTGGTAGAATTCTCG |
| | | (SEQ ID NO: 21) |
| | Reverse | CCGACCCGCAGGCGAGAATTCTACCACTGAACCA (SEQ ID NO: 22) |
| HDV | Forward | TAATACGACTCACTATAGGGCATCTCCACC (SEQ ID NO: 23) |
| ribozyme | Reverse | GAAAAGTGGCTCTCCCTTAGCCATCCGAGTGCTCGGATGCCCAGGTCGG |
| | | ACCGCGAGGAGGTGGAGATGCCC (SEQ ID NO: 24) |

Supplementary Information Table S2

TABLE S2
Sequences and concentrations of TaqMan probes/primers for multiplex
TaqMan RT-qPCR

| | | | | Concentration |
|---|---|---|---|---|
| Group | Target | Probe | Sequence (5′ to 3′) | (nM) |
| 1 | 5′-HisGUG | Forward | GCTCGCCGTGATCGTATAGT (SEQ ID NO: 4) | 200 |
| | | TaqMan | /5HEX/TAGTACTCT/ZEN/GCGTTGGAACACTGCGTTTGC/ | 200 |
| | | | 3IABkFQ/ (SEQ ID NO: 8) | |
| | 5′-GlyCCC | Forward | GCATTGGTGGTTCAGTGGT (SEQ ID NO: 11) | 200 |
| | | TaqMan | /56-TAMN/ATTCTCGCCTGCGAACACTGCG/3IAbRQSp/ | 200 |
| | | | (SEQ ID NO: 14) | |
| | Spike-in* | Forward | GAGGCAAGCCCGACGT (SEQ ID NO: 12) | 200 |
| | | TaqMan | /56-FAM/GATTGTCCG/ZEN/CGAACACTGCGT/3IABkFQ/ | 200 |
| | | | (SEQ ID NO: 15) | |
| | 5S rRNA* | Forward | TACGGCCATACCACCCTGAAC (SEQ ID NO: 13) | 200 |
| | | TaqMan | /56- | 50 |
| | | | FAM/CGGGTGCTG/ZEN/TAGGCTTTGAACACTGCGTT/ | |
| | | | 3IABkFQ/ (SEQ ID NO: 16) | |
| 2 | 5′-LysCUU | Forward | GCCCGGCTAGCTCAG (SEQ ID NO: 5) | 200 |
| | | TaqMan | /5HEX/AGAGCATGG/ZEN/GACTCGAACACTG/3IABkFQ/ | 200 |
| | | | (SEQ ID NO: 9) | |
| | Spike-in* | Forward | GAGGCAAGCCCGACGT (SEQ ID NO: 12) | 200 |
| | | TaqMan | /56-FAM/GATTGTCCG/ZEN/CGAACACTGCGT/3IABkFQ/ | 200 |
| | | | (SEQ ID NO: 15) | |
| | 5S rRNA* | Forward | TACGGCCATACCACCCTGAAC (SEQ ID NO: 13) | 200 |
| | | TaqMan | /56- | 50 |
| | | | FAM/CGGGTGCTG/ZEN/TAGGCTTTGAACACTGCGTT/ | |
| | | | 3IABkFQ/ (SEQ ID NO: 16) | |

Synthetic primers and TaqMan probes disclosed herein were synthesized by Integrated DNA Technologies (the abbreviations within the sequences are according to the company). For TaqMan RT-qPCR, universal reverse primer (5′-GATCGTCGGACTGTAGAACTC-3′) (SEQ ID NO: 2) was used at 600 nM concentration. TaqMan probes contain hexachlorofluorescein (HEX), carboxytetramethylrhodamine (TAMRA), and 6-carboxyfluorescein (FAM) as the fluorophore and ZEN/Iowa Black as the quencher. *As a control, one of either spike-in RNA (for plasma RNAs) or SS rRNA (for cellular RNAs) was quantified. In some embodiments, spike-in RNA and SS rRNA are not quantified simultaneously.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

What is claimed is:

1. A method for performing sequencing of 5′-htRNA in an RNA sample comprising:
obtaining a DNA library of 5′-htRNA by
(a) treating an RNA sample containing 5′-htRNAs having a 3′ cyclic phosphate with a phosphatase;
(b) treating the phosphatase treated RNA sample with a periodate;
(c) treating the periodate treated RNA sample with a polynucleotide kinase;
(d) adding a 3′-RNA adaptor to the RNA sample of step c;
(e) treating the RNA sample of step d with an RNA ligase;
(f) adding a 5′-RNA adaptor to the RNA sample of step e;
(g) treating the RNA sample of step f with an RNA ligase;
(h) performing a RT-PCR on the RNA sample of step g; and sequencing the DNA library of 5′-htRNAs in the RNA sample.

2. The method of claim 1, further comprising enriching one or more 25-55 nt RNA fragments in the RNA sample prior to step (a).

3. The method of claim 1, further comprising gel-purifying one or more 25-55 nt RNA fragments in the RNA sample prior to step (a).

4. The method of claim 1, wherein the 5′-htRNA is 5′-htRNA^{Asp} or 5′-htRNA^{His}.

5. The method of claim 1, wherein the RNA sample is total RNA.

6. The method of claim 1, wherein the RNA sample is derived from a cell, tissue, body fluid, or organ.

7. The method of claim 1, wherein the RNA sample is approximately at least 100 pg.

8. The method of claim 1, wherein the polynucleotide kinase is a T4 polynucleotide kinase.

9. The method of claim 1, wherein the RNA ligase of steps (e) and (g) is a T4 RNA ligase.

10. A DNA library of 5′-htRNAs obtained by the method of claim 1.

11. A method for quantifying 5′-htRNA half molecules in an RNA sample comprising:
(a) treating an RNA sample containing 5′-htRNAs half molecules having a 3′ cyclic phosphate with a T4 polynucleotide kinase to form 3′-dephosphrylated 5′-tRNA halves;
(b) 3′-AD ligating the 3′-dephosphrylated 5′-tRNA halves to form ligated RNA products; wherein treating of step (a) is simultaneously performed as one step in a single tube with the 3′-AD ligating of step (b); and
(c) quantifying the ligated RNA products by RT-qPCR using a plurality of target primers and probes configured to simultaneously quantify at least two cP-containing 5′-tRNA half species.

12. The method of claim 11, wherein quantifying of step (c) simultaneously quantifies 5′-tRNA half species including 5′-tRNA^{LysCUU}, 5′-tRNA^{GluCUC}, 5′-tRNA^{HisGUG}, 5′-tRNA^{GlyCCC}, or combinations thereof.

13. The method of claim 11, wherein quantifying of step (c) further simultaneously quantifies control RNA.

14. The method of claim 13, wherein the control RNA includes spike-in RNA, 5S rRNA, or combinations thereof.

15. The method of claim 11, wherein quantifying of step (c) simultaneously quantifies one of at least three 5′-tRNA half species and at least four 5′-tRNA half species.

16. The method of claim 11, wherein an incubation time for the simultaneous treating and 3′-AD ligating is at most 2 h.

17. The method of claim 16, wherein the incubation time for the simultaneous treating and 3′-AD ligating is at most 1 h at 37° C., followed by at most 1 h at 4° C.

18. The method of claim 11, wherein the RNA sample is total RNA.

19. The method of claim 11, wherein the RNA sample is derived from a cell, tissue, body fluid, or organ.

20. The method of claim 11, wherein the RNA sample is approximately at least 50 ng.
